(19) United States
(12) Patent Application Publication
(10) Pub. No.: US 2009/0118375 A1
(43) Pub. Date: May 7, 2009

(54) Optically active phthalamides

(76) Inventors: Rudiger Fischer, Pulheim (DE); Christian Funke, Leichlingen (DE); Olga Malsam, Rosrath (DE); Peter Losel, Leverkusen-Hitdorf (DE); Ulrich Gorgens, Ratingen (DE); Christian Arnold, Langenfeld (DE); Masanori Tohnischi, Osaka (JP); Minoru Yamaguchi, Osaka (JP); Hiroto Harayama, Osaka (JP); Shinsuke Fujioka, Osaka (JP)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 11/661,091

(22) PCT Filed: Aug. 20, 2005
(86) PCT No.: PCT/EP05/09018

§ 371 Date: Oct. 17, 2008

(30) Foreign Application Priority Data
Aug. 31, 2004 (EP) 04020618.7

(51) Int. Cl. A01N 37/18; C07C 321/20; A01P 7/00; C07C 209/74; C07C 211/52

(52) U.S. Cl. 514/616; 564/154; 564/412; 564/442

(57) ABSTRACT

Novel optically active phthalamides of the formula (I) in which A, q, R¹, R², E¹, E², X, m, Y, n and Z are as defined in the description, processes for preparing these compounds and their use for controlling pests.

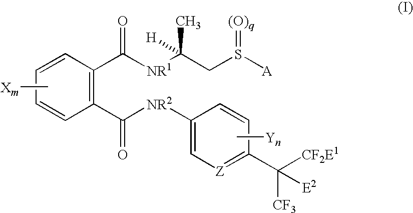

The present invention relates to novel optically active phthalamides, to a plurality of processes for their preparation and to their use as active compounds, in particular their use as pesticides.

It is already known that numerous phthalamides have insecticidal properties (cf. EP-A 0 919 542, EP-A 1 006 107). The activity of these compounds is good; however, it is sometimes unsatisfactory.

This invention now provides novel optically active phthalamides of the formula (I)

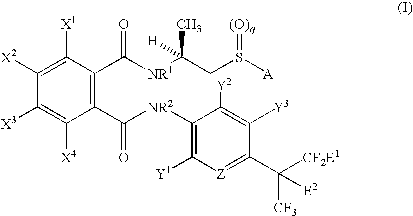

in which
A represents hydrogen, C₁-C₆-alkyl, C₃-C₆-alkenyl, C₃-C₆-alkynyl, C₁-C₆-alkoxy-C₁-C₄-alkyl, C₁-C₆-alkylthio-C₁-C₄-alkyl, C₁-C₆-alkylsulfinyl-C₁-C₄-alkyl, (C₁-C₆-alkyl)carbamoyl,
q represents 0, 1 or 2,
R¹ represents hydrogen or C₁-C₆-alkyl,
R² represents hydrogen or C₁-C₆-alkyl,
Z represents CY⁴ or N,
E¹ represents hydrogen or bromine,
E² represents hydrogen or fluorine,
X¹, X², X³, X⁴ independently represent hydrogen, halogen, C₁-C₆-alkyl, C₁-C₆-haloalkyl, nitro, cyano, C₁-C₆-alkylsulfonyloxy, C₁-C₆-haloalkylsulfonyloxy, phenylsulfonyloxy, C₁-C₆-alkylthio-C₁-C₆-alkyl, C₁-C₆-alkylsulfinyl-C₁-C₆-alkyl, C₁-C₆-alkylsulfonyl-C₁-C₆-alkyl, C₁-C₆-alkylsulfonylamino, bis(C₁-C₆-alkylsulfonyl)amino or C₁-C₆-alkylcarbonyloxy,
Y¹, Y², Y³, Y⁴ independently represent hydrogen, halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio or cyano.

In cases where R¹ represents C₁-C₆-alkyl, R¹ represents hydrogen and in cases where R² represents C₁-C₆-alkyl R¹ represents hydrogen.

Depending on the type and number of substituents, the compounds of the formula (I) may, if appropriate, be present as geometrical and/or optical isomers or regioisomers or isomer mixtures thereof of varying composition. What is claimed by the invention are both the pure isomers and the isomer mixtures.

Furthermore, it has been found that optically active phthalamides of the formula (I) can be prepared according to the following Scheme 1:

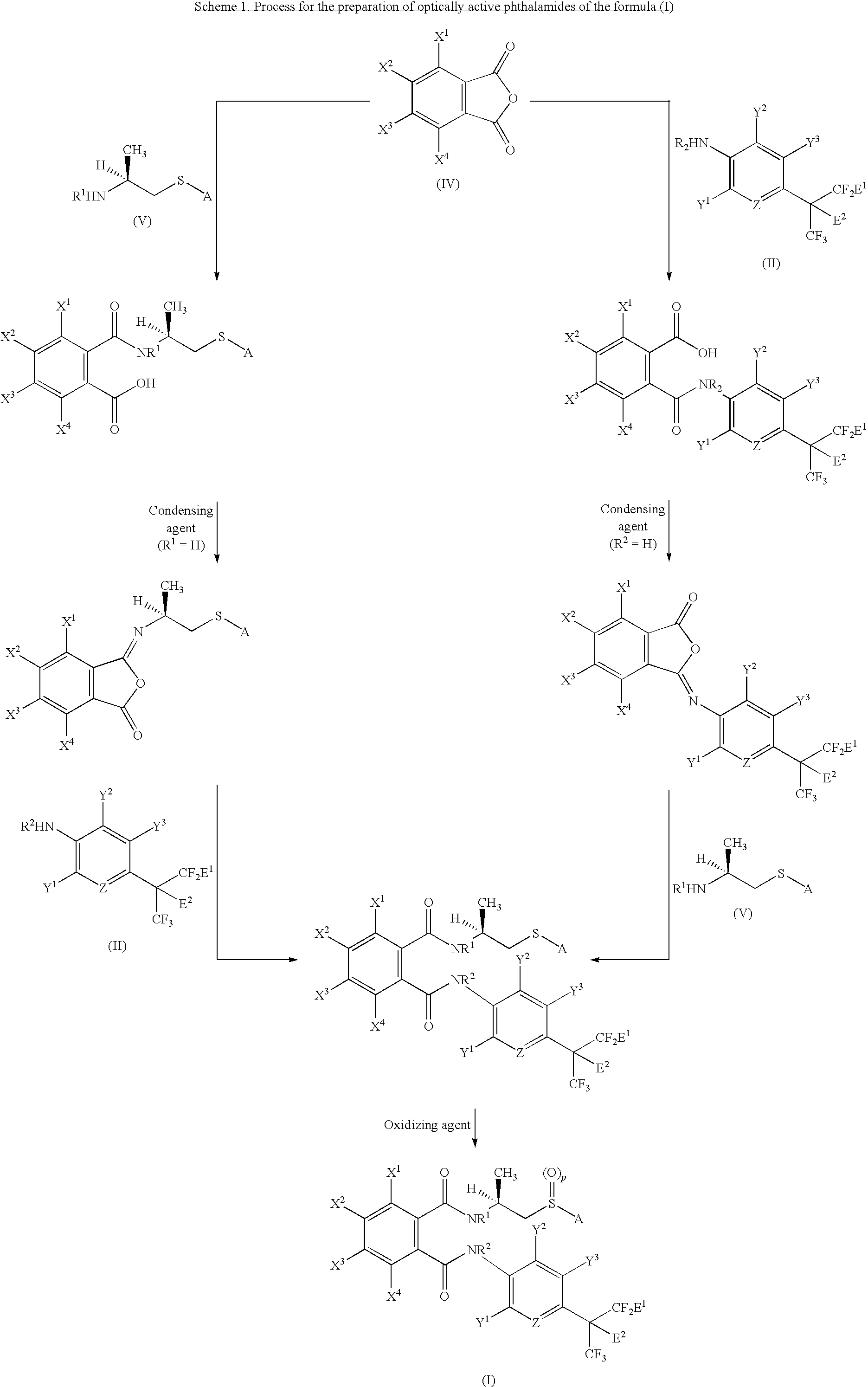

Finally, it has been found that the compounds of the formula (I) according to the invention have very good insecticidal properties and can be used both in crop protection and in the protection of materials for controlling unwanted pests, such as insects, arachnids and mites.

The formula (I) provides a general definition of the compounds according to the invention. Preferred substituents and ranges of the radicals given in the formulae mentioned above and below are illustrated below.
A preferably represents hydrogen, methyl, ethyl, n- or isopropyl, n-, iso-, sec- or tert-butyl, the isomeric pentyls, the isomeric hexyls, allyl, butenyl, pentenyl, hexenyl, propargyl, butynyl, pentynyl, hexynyl, C₁-C₄-alkoxy-C₁-C₂-alkyl, C₁-C₄-alkylthio-C₁-C₂-alkyl, C₁-C₄-alkylsulfinyl-C₁-C₂-alkyl, (C₁-C₄-alkyl)carbamoyl.
A particularly preferably represents hydrogen, methyl, ethyl, n- or isopropyl, n-, iso-, sec- or tert-butyl, allyl, butenyl, propargyl, butynyl, C₁-C₄-alkoxy-C₁-C₂-alkyl (such as e.g. methoxy-methyl, methoxyethyl, ethoxymethyl, ethoxyethyl, n- or isopropoxymethyl), C₁-C₄-alkylthio-C₁-C₂-alkyl (such as e.g. methylthiomethyl, methylthioethyl, ethylthiomethyl, ethylthioethyl, n- or isopropylthiomethyl), C₁-C₄-alkylsulfinyl-C₁-C₂-alkyl (such as e.g. methylsulfinylmethyl, methylsulfinylethyl, ethylsulfinylmethyl, ethylsulfinylethyl, n- or isopropylsulfinylmethyl).
A very particularly preferably represents methyl, ethyl, n- or isopropyl, n-, iso-, sec- or tert-butyl, allyl, butenyl, propargyl, butynyl.
A especially preferably represents methyl, ethyl, n- or isopropyl.
q preferably represents 0.
q furthermore preferably represents 1.
q furthermore preferably represents 2.
R¹ preferably represents hydrogen, methyl, ethyl, n- or isopropyl, n-, iso-, sec- or tert-butyl.
R¹ particularly preferably represents hydrogen, methyl, ethyl, isopropyl or tert-butyl.
R¹ very particularly preferably represents hydrogen.
R² preferably represents hydrogen, methyl, ethyl, n- or isopropyl, n-, iso-, sec- or tert-butyl.
R² particularly preferably represents hydrogen, methyl, ethyl, isopropyl or tert-butyl.
R² very particularly preferably represents hydrogen.
Z preferably represents CY⁴.
Z furthermore preferably represents N.
E¹ preferably represents hydrogen.
E¹ furthermore preferably represents bromine.
E² preferably represents hydrogen.
E² furthermore preferably represents fluorine.
X¹, X², X³, X⁴ independently preferably represent hydrogen, fluorine, chlorine, bromine, iodine, C₁-C₄-alkyl, C₁-C₄-haloalkyl, nitro, cyano, C₁-C₄-alkylsulfonyloxy, C₁-C₄-haloalkylsulfonyloxy; phenylsulfonyloxy, C₁-C₄-alkylthio-C₁-C₄-alkyl, C₁-C₄-alkylsulfonyloxy-C₁-C₄-alkyl, C₁-C₄-alkylsulfonyl-C₁-C₄-alkyl, C₁-C₄-alkylsulfonylamino, bis(C₁-C₄-alkylsulfonyl)amino or C₁-C₄-alkylcarbonyloxy.
X¹, X², X³, X⁴ independently particularly preferably represent hydrogen, fluorine, chlorine, bromine, iodine, C₁-C₄-alkyl (such as e.g. methyl, ethyl, n- or isopropyl, n-, iso, sec- or tert-butyl), C₁-C₄-haloalkyl (such as e.g. trifluoromethyl, trifluoroethyl, trichloromethyl), nitro, cyano, C₁-C₄-alkylsulfonyloxy (such as e.g. methylsulfonyloxy, ethylsulfonyloxy, n- or isopropylsulfonyloxy, n-, iso-, sec- or tert-butylsulfonyloxy), C₁-C₄-haloalkylsulfonyloxy,
X¹ very particularly preferably represents fluorine, chlorine, bromine, iodine, C₁-C₄-alkyl-sulfonyloxy (such as e.g. methylsulfonyloxy, ethylsulfonyloxy, n- or isopropylsulfonyloxy).
X², X³, X⁴ independently very particularly preferably represent hydrogen.
Y¹, Y², Y³, Y⁴ independently preferably represent hydrogen, fluorine, chlorine, bromine, iodine, C₁-C₄-alkyl, C₁-C₄-haloalkyl, C₁-C₄-alkoxy, C₁-C₄-haloalkoxy, C₁-C₄-alkylthio, C₁-C₄-haloalkyl-thio, or cyano.
Y¹, Y², Y³, Y⁴ independently particularly preferably represent hydrogen, fluorine, chlorine, bromine, iodine, C₁-C₄-alkyl (such as e.g. methyl, ethyl, n- or isopropyl, n-, iso, sec- or tert-butyl), C₁-C₄-haloalkyl (such as e.g. trifluoromethyl, trifluoroethyl, trichloromethyl), C₁-C₄-alkoxy (such as e.g. methoxy, ethoxy, n- or isopropoxy, n-, iso, sec- or tert-butoxy), C₁-C₄-halo-alkoxy (such as e.g. trifluoromethoxy, trifluoroethoxy, trichloromethoxy).
Y¹ very particularly preferably represents fluorine, chlorine, bromine, methyl, ethyl.
Y², Y³, Y⁴ independently very particularly preferably represent hydrogen.

In cases where R¹ is methyl, ethyl, n- or isopropyl, n-, iso-, sec- or tert-butyl, R² represents hydrogen.

In cases where R² is methyl, ethyl, n- or isopropyl, n-, iso-, sec- or tert-butyl, R¹ represents hydrogen.

Preference is furthermore given to compounds of the formula (I) in which R¹ and R² each represent hydrogen.

Preference is furthermore given to compounds of the formula (I) in which X², X³ and X⁴ each represent hydrogen.

Preference is furthermore given to compounds of the formula (I) in which Y², Y³ and Y⁴ each represent hydrogen.

Saturated hydrocarbon radicals, such as alkyl, can in each case be straight-chain or branched as far as this is possible—including in combination with heteroatoms, such as, for example, in alkoxy.

Halogen-substituted radicals, for example haloalkyl, are mono- or polysubstituted, up to the maximum number of substituents possible. In the case of polyhalogenation, the halogen atoms can be identical or different. Here, halogen represents fluorine, chlorine, bromine or iodine, in particular fluorine, chlorine or bromine.

Preference, particular preference and very particular preference is given to compounds carrying the substituents mentioned under preferred, particularly preferred and very particularly preferred, respectively.

Saturated or unsaturated hydrocarbon radicals, such as alkyl or alkenyl, can in each case be straight-chain or branched as far as this is possible—including in combination with heteroatoms, such as, for example, in alkoxy.

Optionally substituted radicals may be mono- or polysubstituted, where in the case of polysubstitution the substituents may be identical or different.

However, the general or preferred radical definitions or illustrations given above can also be combined with one another as desired, i.e. between the respective ranges and preferred ranges. They apply to the end products and, correspondingly, to the precursors and intermediates.

The preparation of the optically active phthalamides of the formula (I) according to the invention is depicted in Scheme 1. Reaction conditions for the single reaction steps are identical to the reactions described in EP-A 0 919 542 and EP-A 1 006 107 (cf. also the preparation examples below).

Compounds of the formula (I) may be obtained furthermore by synthesis of the racemic analogues which racemates are subsequently resolved by customary methods, such as, for example, by chromatography of the corresponding racemates on a chiral stationary phase. In this manner, it is possible to resolve both the racemic end products or racemic intermediates into the two enantiomers.

Compounds of the formula (II) (cf. Scheme 1)

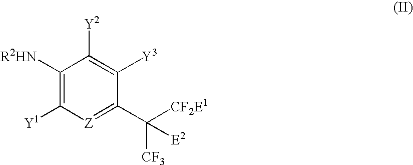

can be obtained as depicted in the following Scheme 2

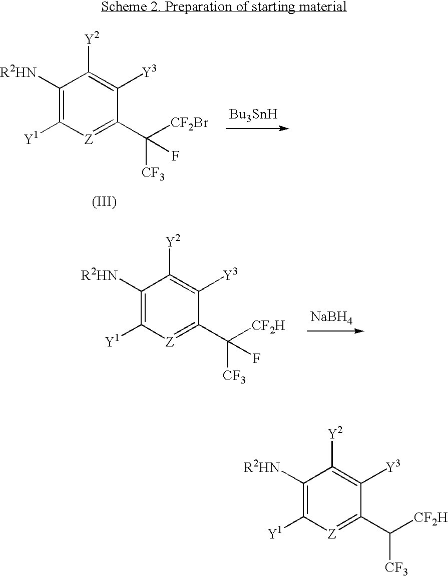

The compounds of formula (III) may be obtained according to processes described in EP-A 1 418 169 and EP-A 1 418 163.

Compounds of formula (IV) are either commercially available or can be prepared according to JP 2001335571.

Compounds of formula (V) can be obtained by the process described in U.S. Pat. No. 6,639,109.

The active compounds are suitable for protecting plants and plant organs, for increasing the harvest yields, for improving the quality of the harvested material and for controlling animal pests, in particular insects, arachnids and nematodes, which are encountered in agriculture, in forests, in gardens and leisure facilities, in the protection of stored products and of materials, and in the hygiene sector, and have good plant tolerance and favourable toxicity to warm-blooded animals and are tolerated well by the environment. They may be preferably employed as plant protection agents. They are active against normally sensitive and resistance species and against all or some stages of development. The abovementioned pests include:

From the order of the Isopoda, for example, Oniscus asellus, Armadillidium vulgare and Porcellio scaber.
From the order of the Diplopoda, for example, Blaniulus guttulatus.
From the order of the Chilopoda, for example, Geophilus carpophagus and Scutigera spp.
From the order of the Symphyla, for example, Scutigerella immaculate.
From the order of the Thysanura, for example, Lepisma saccharina.
From the order of the Collembola, for example, Onychiurus armatus.
From the order of the Orthoptera, for example, Acheta domesticus, Gryllotalpa spp., Locusta migratoria migratorioides, Melanoplus spp. and Schistocerca gregaria.
From the order of the Blattaria, for example, Blatta orientalis, Periplaneta americana, Leucophaea maderae, Blattella germanica.
From the order of the Dermaptera, for example, Forficula auricularia.
From the order of the Isoptera, for example, Reticulitermes spp.
From the order of the Phthiraptera, for example, Pediculus humanus corporis, Haematopinus spp., Linognathus spp., Trichodectes spp. and Damalinia spp.
From the order of the Thysanoptera, for example, Hercinothrips femoralis, Thrips tabaci, Thrips palmi and Frankliniella occidentalis.
From the order of the Heteroptera, for example, Eurygaster spp., Dysdercus intermedius, Piesma quadrata, Cimex lectularius, Rhodnius prolixus and Triatoma spp.
From the order of the Homoptera, for example, Aleurodes brassicae, Bemisia tabaci, Trialeurodes vaporariorum, Aphis gossypii, Brevicoryne brassicae, Cryptomyzus ribis, Aphis fabae, Aphis pomi, Eriosoma lanigerum, Hyalopterus arundinis, Phylloxera vastatrix, Pemphigus spp., Macrosiphum avenae, Myzus spp., Phorodon humuli, Rhopalosiphum padi, Empoasca spp., Euscelis bilobatus, Nephotettix cincticeps, Lecanium corni, Saissetia oleae, Laodelphax striatellus, Nilaparvata lugens, Aonidiella aurantii, Aspidiotus hederae, Pseudococcus spp. and Psylla spp.
From the order of the Lepidoptera, for example, Pectinophora gossypiella, Bupalus piniarius, Chematobia brumata, Lithocolletis blancardella, Hyponomeuta padella, Plutella xylostella, Malacosoma neustria, Euproctis chrysorrhoea, Lymantria spp., Bucculatrix thurberiella, Phyllocnistis citrella, Agrotis spp., Euxoa spp., Feltia spp., Earias insulana, Heliothis spp., Mamestra brassicae, Panolis flammea, Spodoptera spp., Trichoplusia ni, Carpocapsa pomonella, Pieris spp., Chilo spp., Pyrausta nubilalis, Ephestia kuehniella, Galleria mellonella, Tineola bisselliella, Tinea pellionella, Hofmannophila pseudospretella, Cacoecia podana, Capua reticulana, Choristoneura fumiferana, Clysia ambiguella, Homona magnanima, Tortrix viridana, Cnaphalocerus spp., Oulema oryzae.
From the order of the Coleoptera, for example, Anobium punctatum, Rhizopertha dominica, Bruchidius obtectus, Acanthoscelides obtectus, Hylotrupes bajulus, Agelastica alni, Leptinotarsa decemlineata, Phaedon cochleariae, Diabrotica spp., Psylliodes chrysocephala, Epilachna varivestis, Atomaria spp., Oryzaephilus surinamensis, Anthonomus spp., Sitophilus spp., Otiorrhynchus sulcatus, Cosmopolites sordidus, Ceuthorrhynchus assimilis, Hypera postica, Dermestes spp., Trogoderma spp., Anthrenus spp., Attagenus spp., Lyctus spp., Meligethes aeneus, Ptinus spp., Niptus hololeucus, Gibbium psylloides, Tribolium spp., Tenebrio molitor, Agriotes spp., Conoderus spp., Melolontha melolontha, Amphimallon solstitialis, Costelytra zealandica and Lissorhoptrus oryzophilus.
From the order of the Hymenoptera, for example, Diprion spp., Hoplocampa spp., Lasius spp., Monomorium pharaonis and Vespa spp.
From the order of the Diptera, for example, Aedes spp., Anopheles spp., Culex spp., Drosophila melanogaster, Musca spp., Fannia spp., Calliphora erythrocephala, Lucilia spp., Chrysomyia spp., Cuterebra spp., Gastrophilus spp., Hyppobosca spp., Stomoxys spp., Oestrus spp., Hypoderma spp., Tabanus spp., Tannia spp., Bibio hortulanus, Oscinella frit, Phorbia spp., Pegomyia hyoscyami, Ceratitis capitata, Dacus oleae, Tipula paludosa, Hylemyia spp. and Liriomyza spp.
From the order of the Siphonaptera, for example, Xenopsylla cheopis and Ceratophyllus spp.
From the class of the Arachnida, for example, Scorpio maurus, Latrodectus mactans, Acarus siro, Argas spp., Ornithodoros spp., Dermanyssus gallinae, Eriophyes ribis, Phyllocoptruta oleivora, Boophilus spp., Rhipicephalus spp., Amblyomma spp., Hyalomma spp., Ixodes spp., Psoroptes spp., Chorioptes spp., Sarcoptes spp., Tarsonemus spp., Bryobia praetiosa, Panonychus spp., Tetranychus spp., Hemitarsonemus spp., Brevipalpus spp.

The phytoparasitic nematodes include, for example, Pratylenchus spp., Radopholus similis, Ditylenchus dipsaci, Tylenchulus semipenetrans, Heterodera spp., Globodera spp., Meloidogyne spp., Aphelenchoides spp., Longidorus spp., Xiphinema spp., Trichodorus spp., Bursaphelenchus spp.

The compounds of the formula (I) according to the invention have in particular excellent activity against caterpillars and beetle larvae.

If appropriate, the compounds according to the invention can, at certain concentrations or application rates, also be used as herbicides or microbicides, for example as fungicides, antimycotics and bactericides. If appropriate, they can also be employed as intermediates or precursors for the synthesis of other active compounds.

All plants and plant parts can be treated in accordance with the invention. Plants are to be understood as meaning in the present context all plants and plant populations such as desired and undesired wild plants or crop plants (including naturally occurring crop plants). Crop plants can be plants which can be obtained by conventional plant breeding and optimization methods or by biotechnological and genetic engineering methods or by combinations of these methods, including the transgenic plants and including the plant cultivars protectable or not protectable by plant breeders' rights. Plant parts are to be understood as meaning all parts and organs of plants above and below the ground, such as shoot, leaf, flower and root, examples which may be mentioned being leaves, needles, stalks, stems, flowers, fruit bodies, fruits, seeds, roots, tubers and rhizomes. The plant parts also include harvested material, and vegetative and generative propagation material, for example cuttings, tubers, rhizomes, offshoots and seeds.

Treatment according to the invention of the plants and plant parts with the active compounds is carried out directly or by allowing the compounds to act on the surroundings, environment or storage space by the customary treatment methods, for example by immersion, spraying, evaporation, fogging, scattering, painting on and, in the case of propagation material, in particular in the case of seeds, also by applying one or more coats.

The active compounds can be converted to the customary formulations, such as solutions, emulsions, wettable powders, suspensions, powders, dusts, pastes, soluble powders, granules, suspension-emulsion concentrates, natural and synthetic materials impregnated with active compound and microencapsulations in polymeric substances.

These formulations are produced in a known manner, for example by mixing the active compounds with extenders, that is liquid solvents, and/or solid carriers, optionally with the use of surfactants, that is emulsifiers and/or dispersants, and/or foam-formers.

If the extender used is water, it is also possible to employ for example organic solvents as auxiliary solvents. Essentially, suitable liquid solvents are: aromatics such as xylene, toluene or alkylnaphtha-lenes, chlorinated aromatics or chlorinated aliphatic hydrocarbons such as chlorobenzenes, chloro-ethylenes or methylene chloride, aliphatic hydrocarbons such as cyclohexane or paraffins, for example petroleum fractions, mineral and vegetable oils, alcohols such as butanol or glycol and also their ethers and esters, ketones such as acetone, methyl ethyl ketone, methyl isobutyl ketone or cyclo-hexanone, strongly polar solvents such as dimethylformamide and dimethyl sulphoxide, and also water.

As solid carriers there are suitable:

for example ammonium salts and ground natural minerals such as kaolins, clays, talc, chalk, quartz, attapulgite, montmorillonite or diatomaceous earth, and ground synthetic minerals, such as highly disperse silica, alumina and silicates; as solid carriers for granules there are suitable: for example crushed and fractionated natural rocks such as calcite, marble, pumice, sepiolite and dolomite, and also synthetic granules of inorganic and organic meals, and granules of organic material such as sawdust, coconut shells, maize cobs and tobacco stalks; as emulsifiers and/or foam-formers there are suitable:
for example nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates and also protein hydrolysates; as dispersants there are suitable: for example lignosulphite waste liquors and methylcellulose.

Tackifiers such as carboxymethylcellulose and natural and synthetic polymers in the form of powders, granules or latices, such as gum arabic, polyvinyl alcohol and polyvinyl acetate, as well as natural phospholipids such as cephalins and lecithins, and synthetic phospholipids, can be used in the formulations. Other additives can be mineral and vegetable oils.

It is possible to use colorants such as inorganic pigments, for example iron oxide, titanium oxide and Prussian Blue, and organic dyestuffs, such as alizarin dyestuffs, azo dyestuffs and metal phthalocyanine dyestuffs, and trace nutrients such as salts of iron, manganese, boron, copper, cobalt, molybdenum and zinc.

The formulations generally comprise between 0.1 and 95% by weight of active compound, preferably between 0.5 and 90%.

The active compound according to the invention can be used in its commercially available formulations and in the use forms, prepared from these formulations, as a mixture with other active compounds, such as insecticides, attractants, sterilizing agents, bactericides, acaricides, nematicides, fungicides, growth-regulating substances or herbicides. The insecticides include, for example, phosphoric acid esters, carbamates, carboxylates, chlorinated hydrocarbons, phenylureas and substances produced by microorganisms, inter alia.

Particularly favourable mixing components are, for example, the following compounds:

### Fungicides:

2-phenylphenol; 8-hydroxyquinoline sulfate; acibenzolar-S-methyl; aldimorph; amidoflumet; ampro-pylfos; ampropylfos-potassium; andoprim; anilazine; azaconazole; azoxystrobin; benalaxyl; benalaxyl-M; benodanil; benomyl; benthiavalicarb-isopropyl; benzamacril; benzamacril-isobutyl; bilanafos; bina-pacryl; biphenyl; bitertanol; blasticidin-S; boscalid; bromuconazole; bupirimate; buthiobate; butyl-amine; calcium polysulfide; capsimycin; captafol; captan; carbendazim; carboxin; carpropamid; carvone; chinomethionat; chlobenthiazone; chlorfenazole; chloroneb; chlorothalonil; chlozolinate; clozylacon; cyazofamid; cyflufenamid; cymoxanil; cyproconazole; cyprodinil; cyprofuram; Dagger G; debacarb; dichlofluanid; dichlone; dichlorophen; diclocymet; diclomezine; dicloran; diethofencarb; difenoconazole; diflumetorim; dimethirimol; dimethomorph; dimoxystrobin; diniconazole; diniconazole-M; dinocap; diphenylamine; dipyrithione; ditalimfos; dithianon; dodine; drazoxolon; edifenphos; epoxiconazole; ethaboxam; ethirimol; etridiazole; famoxadone; fenamidone; fenapanil; fenarimol; fenbuconazole; fenfuram; fenhexamid; fenitropan; fenoxanil; fenpiclonil; fenpropidin; fenpropimorph; ferbam; fluazinam; flubenzimine; fludioxonil; flumetover; flumorph; fluoromide; fluoxastrobin; fluquinconazole; flurprimidol; flusilazole; flusulfamide; flutolanil; flutriafol; folpet; fosetyl-Al; fosetyl-sodium; fuberidazole; furalaxyl; furametpyr; furcarbanil; furmecyclox; guazatine; hexachlorobenzene; hexaconazole; hymexazol; imazalil; imibenconazole; iminoctadine triacetate; iminoctadine tris(albesilate); iodocarb; ipconazole; iprobenfos; iprodione; iprovalicarb; irumamycin; isoprothiolane; isovaledione; kasugamycin; kresoxim-methyl; mancozeb; maneb; meferimzone; mepanipyrim; mepronil; metalaxyl; metalaxyl-M; metconazole; methasulfocarb; methfuroxam; metiram; metominostrobin; metsulfovax; mildiomycin; myclobutanil; myclozolin; natamycin; nicobifen; nitrothal-isopropyl; noviflumuron; nuarimol; ofurace; orysastrobin; oxadixyl; oxolinic acid; oxpoconazole; oxycarboxin; oxyfenthiin; paclobutrazol; pefurazoate; penconazole; pencycuron; phosdiphen; phthalide; picoxystrobin; piperalin; polyoxins; polyoxorim; probenazole; prochloraz; procymidone; propamocarb; propanosine-sodium; propiconazole; propineb; proquinazid; prothioconazole; pyraclostrobin; pyrazophos; pyrifenox; pyrimethanil; pyroquilon; pyroxyfur; pyrrolnitrine; quinconazole; quinoxyfen; quintozene; simeconazole; spiroxamine; sulfur; tebuconazole; tecloftalam; tecnazene; tetcyclacis; tetraconazole; thiabendazole; thicyofen; thifluzamide; thiophanate-methyl; thiram; tioxymid; tolclofos-methyl; tolylfluanid; triadimefon; triadimenol; triazbutil; triazoxide; tricyclamide; tricyclazole; tridemorph; trifloxystrobin; triflumizole; triforine; triticonazole; uniconazole; validamycin A; vinclozolin; zineb; ziram; zoxamide; (2S)-N-[2-[4-[[3-(4-chlorophenyl)-2-propynyl]oxy]-3-methoxyphenyl]ethyl]-3-methyl-2-[(methylsulfonyl)amino]-butanamide; 1-(1-naphthalenyl)-1H-pyrrol-2,5-dione; 2,3,5,6-tetrachloro-4-(methylsulfonyl)-pyridine; 2-amino-4-methyl-N-phenyl-5-thiazolecarboxamide; 2-chloro-N-(2,3-dihydro-1,1,3-trimethyl-1H-inden-4-yl)-3-pyridinecarboxamide; 3,4,5-trichloro-2,6-pyridinedicarbonitrile; actinovate; cis-1-(4-chlorophenyl)-2-(1H-1,2,4-triazol-1-yl)-cycloheptanol; methyl 1-(2,3-dihydro-2,2-dimethyl-1H-inden-1-yl)-1H-imidazole-5-carboxylate; mono potassium carbonate; N-6-methoxy-3-pyridinyl)-cyclopropane-carboxamide; N-butyl-8-(1,1-dimethylethyl)-1-oxaspiro[4.5]decan-3-amine; sodium tetrathiocarbonate; and copper salts and preparations, such as Bordeaux mixture; copper hydroxide; copper naphthenate; copper oxychloride; copper sulphate; cufraneb; copper oxide; mancopper; oxine-copper.

### Bactericides:

bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulphate and other copper preparations.

### Insecticides/Acaricides/Nematicides:

### 1. Acetylcholinesterase (AChE) Inhibitors

1.1 Carbamates (e.g. alanycarb, aldicarb, aldoxycarb, allyxycarb, aminocarb, azamethiphos, bendiocarb, benfuracarb, bufencarb, butacarb, butocarboxim, butoxycarboxim, carbaryl, carbofuran, carbosulfan, chloethocarb, coumaphos, cyanofenphos, cyanophos, dimetilan, ethiofencarb, fenobucarb, fenothiocarb, formetanate, furathiocarb, isoprocarb, metam-sodium, methiocarb, methomyl, metolcarb, oxamyl, pirimicarb, promecarb, propoxur, thiodicarb, thiofanox, triazamate, trimethacarb, XMC, xylylcarb)
1.2 Organophosphates (e.g. acephate, azamethiphos, azinphos (-methyl, -ethyl), bromophos-ethyl, bromfenvinfos (-methyl), butathiofos, cadusafos, carbophenothion, chlorethoxyfos, chlorfenvinphos, chlormephos, chlorpyrifos (-methyl/-ethyl), coumaphos, cyanofenphos, cyanophos, chlorfenvinphos, demeton-s-methyl, demeton-s-methylsulphon, dialifos, diazinon, dichlofenthion, dichlorvos/ddvp, dicrotophos, dimethoate, dimethylvinphos, dioxabenzofos, disulfoton, epn, ethion, ethopropbos, etrimfos, famphur, fenamiphos, fenitrothion, fensulfothion, fenthion, flupyrazofos, fonofos, formothion, fosmethilan, fosthiazate, heptenophos, iodofenphos, iprobenfos, isazofos, isofenphos, isopropyl o-salicylate, isoxathion, malathion, mecarbam, methacrifos, methamidophos, methidathion, mevinphos, monocrotophos, naled, omethoate, oxydemeton-methyl, parathion (-methyl/-ethyl), phenthoate, phorate, phosalone, phosmet, phosphamidon, phosphocarb, phoxim, pirimiphos (-methyl/-ethyl), profenofos, propaphos, propetamphos, prothiofos, prothoate, pyraclofos, pyridaphenthion, pyridathion, quinalphos, sebufos, sulfotep, sulprofos, tebupirimfos, temephos, terbufos, tetrachlorvinphos, thiometon, triazophos, triclorfon, vamidothion)

### 2. Sodium Channel Modulators/Voltage Dependant Sodium Channel Blockers

2.1 Pyrethroids (e.g. acrinathrin, allethrin (d-cis-trans, d-trans), beta-cyfluthrin, bifenthrin, bioallethrin, bioallethrin-s-cyclopentyl-isomer, bioethanomethrin, biopermethrin, bioresmethrin, chlovaporthrin, cis-cypermethrin, cis-resmethrin, cis-permethrin, clocythrin, cycloprothrin, cyfluthrin, cyhalothrin, cypermethrin (alpha-, beta-, theta-, zeta-), cyphenothrin, DDT, deltamethrin, empenthrin (1R-isomer), esfenvalerate, etofenprox, fenfluthrin, fenpropathrin, fenpyrithrin, fenvalerate, flubrocythrinate, flucythrinate, flufenprox, flumethrin, fluvalinate, fubfenprox, gamma-cyhalothrin, imiprothrin, kadethrin, lambda-cyhalothrin, metofluthrin, permethrin (cis-, trans-), phenothrin (1R-trans isomer), prallethrin, profluthrin, protrifenbute, pyresmethrin, resmethrin, RU 15525, silafluofen, tau-fluvalinate, tefluthrin, terallethrin, tetramethrin (1R-isomer), tralomethrin, transfluthrin, ZXI 8901, pyrethrins (pyrethrum))
2.2 Oxadiazine (e.g. indoxacarb)

### 3. Acetylcholine Receptor Agonists/-Antagonists

3.1 Chloronicotinyls/neonicotinoids (e.g. acetamiprid, clothianidin, dinotefuran, imidacloprid, nitenpyram, nithiazine, thiacloprid, thiamethoxam)
3.2 nicotine, bensultap, cartap

### 4. Acetylcholine Receptor Modulators

4.1 Spinosyns (e.g. spinosad)

### 5. GABA Gated Chloride Channel Antagonists

5.1 Cyclodiene organochlorines (e.g. camphechlor, chlordane, endosulfan, ganma-HCH, HCH, heptachlor, lindane, methoxychlor
5.2 Fiproles (e.g. acetoprole, ethiprole, fipronil, vaniliprole)

### 6. Chloride Channel Activators

6.1 Mectins (e.g. abamectin, avermectin, emamectin, emamectin-benzoate, ivermectin, milbemectin, milbemycin)

### 7. Juvenile Hormone Mimics

(e.g. diofenolan, epofenonane, fenoxycarb, hydroprene, kinoprene, methoprene, pyriproxifen, triprene)

### 8. Ecdysone Agonists/Disruptors

8.1 Diacylhydrazines (e.g. chromafenozide, halofenozide, methoxyfenozide, tebufenozide)

### 9. Inhibitors of Chitin Biosynthesis

9.1 Benzoylureas (e.g. bistrifluoron, chlofluazuron, diflubenzuron, fluazuron, flucycloxuron, flufenoxuron, hexaflumuron, lufenuron, novaluron, noviflumuron, penfluoron, teflubenzuron, triflumuron)
9.2 buprofezin
9.3 cyromazine

### 10. Inhibitors of Oxidative Phosphorylation, ATP-Disruptors

10.1 diafenthiuron
10.2 Organotins (e.g. azocyclotin, cyhexatin, fenbutatin-oxide)

### 11. Decoupler Oxidative Phoshorylation by Diruption of H Proton Gradient

11.1 Pyrroles (e.g. chlorfenapyr)
11.2 Dinitrophenoles (e.g. binapacyrl, dinobuton, dinocap, DNOC)

### 12. Site I Electron Transport Inhibitors

12.1 METI's (e.g. fenazaquin, fenpyroximate, pyrimidifen, pyridaben, tebufenpyrad, tolfenpyrad)
12.2 hydramethylnone
12.3 dicofol

### 13. Site II Electron Transport Inhibitors

13.1 rotenone

### 14. Site III Electron Transport Inhibitors

14.1 acequinocyl, fluacrypyrim

### 15. Microbial Disruptors of Insect Midgut Membranes

Bacillus thuringiensis strains

### 16. Inhibitors of Lipid Synthesis

16.1 Tetronic acid insecticides (e.g. spirodiclofen, spiromesifen)
16.2 Tetramic acid insecticides [e.g. 3-(2,5-dimethylphenyl)-8-methoxy-2-oxo-1-azaspiro(4.5]dec-3-en-4-yl ethyl carbonate (alias: carbonic acid, 3-(2,5-dimethylphenyl)-8-methoxy-2-oxo-1-azaspiro-[4.5]dec-3-en-4-yl ethyl ester, CAS-Reg.-No.: 382608-10-08) and carbonic acid, cis-3-(2,5-dimethyl-phenyl)-8-methoxy-2-oxo-1-azaspiro[4.5]dec-3-en-4-yl ethyl ester (CAS-Reg.-No.: 203313-25-1)]

### 17 Carboxamides

(e.g. flonicamid)

### 18. Octopaminergic Agonists

(e.g. amitraz)

### 19. Inhibitors of Magnesium Stimulated ATPase

(e.g. propargite)

### 20. Phthalamides

(e.g. N²-[1,1-dimethyl-2-(methylsulfonyl)ethyl]-3-iodo-N¹-[2-methyl-4-[1,2,2,2-tetrafluoro-1-(tri-fluoromethyl)ethyl]phenyl]-1,2-benzenedicarboxamide (CAS-Reg.-No.: 272451-65-7, fluben-diamide))

### 21. Nereistoxin Analogues

(e.g. thiocyclam hydrogen oxalate, thiosultap-sodium)

### 22. Biologica, Hormones or Pheromones

(e.g. azadirachtin, Bacillus spec., Beauveria spec., codlemone, Metarrhizium spec., Paecilomyces spec., thuringiensin, Verticillium spec.)

### 23. Compounds of Unknown or Non-Specific Mode of Action

23.1 Fumigants (e.g. aluminium phosphide, methyl bromide, sulfuryl fluoride)
23.2 Selective feeding blockers (e.g. cryolite, flonicamid, pymetrozine)
23.3 Mite growth inhibitors (e.g. clofentezine, etoxazole, hexythiazox)
23.4 amidoflumet, benclothiaz, benzoximate, bifenazate, bromopropylate, buprofezin, chinomethionat, chlordimeform, chlorobenzilate, chloropicrin, clothiazoben, cycloprene, cyflumetofen, dicyclanil, fenoxacrim, fentrifanil, flubenzimine, flufenerim, flutenzin, gossyplure, hydramethylnone, japonilure, metoxadiazone, petroleum, piperonyl butoxide, potassium oleate, pyrafluprole, pyridalyl, pyriprole, sulfluramid, tetradifon, tetrasul, triarathene, verbutin
further the compound 3-methyl-phenyl-propylcarbamate (Tsumacide Z), the compound 3-(5-chloro-3-pyridinyl)-8-(2,2,2-trifluoroethyl)-8-azabicyclo[3.2.1]octan-3-carbonitrile (CAS-Reg.-Nr. 185982-80-3) and the corresponding 3-endo isomer (CAS-Reg.-Nr. 185984-60-5) (cf. WO 96/37494, WO 98/25923), and preparations comprising insecticidal active plant extracts, nematodes, fungi or viruses.

A mixture with other known active compounds, such as herbicides, fertilizers, growth regulators, safeners and/or semiochemicals is also possible.

When used as insecticides, the active compounds according to the invention can furthermore be present in their commercially available formulations and in the use forms, prepared from these formulations, as a mixture with synergistic agents. Synergistic agents are compounds, which increase the action of the active compounds, without it being necessary for the synergistic agent added to be active itself.

When used as insecticides, the active compounds according to the invention can furthermore be present in their commercially available formulations and in the use forms, prepared from these formulations, as a mixture with inhibitors which reduce degradation of the active compound after use in the vicinity of the plant, on the surface of parts of plants or in plant tissues.

The active compound content of the use forms prepared from the commercially available formulations can vary within wide limits. The active compound concentration of the use forms can be from 0.0000001 to 95% by weight of active compound, preferably between 0.0001 and 1% by weight.

The compounds are employed in a customary manner appropriate for the use forms.

When used against hygiene pests and pests of stored products, the active compound is distinguished by an excellent residual action on wood and clay as well as a good stability to alkali on limed substrates.

As already mentioned above, it is possible to treat all plants and their parts according to the invention. In a preferred embodiment, wild plant species and plant cultivars, or those obtained by conventional biological breeding methods, such as crossing or protoplast fusion, and parts thereof, are treated. In a further preferred embodiment, transgenic plants and plant cultivars obtained by genetic engineering, if appropriate in combination with conventional methods (Genetically Modified Organisms), and parts thereof are treated. The term “parts” or “parts of plants” or “plant parts” has been explained above.

Particularly preferably, plants of the plant cultivars which are in each case commercially available or in use are treated according to the invention. Plant cultivars are to be understood as meaning plants having certain properties (“traits”) which have been obtained by conventional breeding, by mutagenesis or by recombinant DNA techniques. These can be cultivars, bio- or genotypes.

Depending on the plant species or plant cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the substances and compositions to be used according to the invention, better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products are possible which exceed the effects which were actually to be expected.

The transgenic plants or plant cultivars (i.e. those obtained by genetic engineering) which are preferably to be treated according to the invention include all plants which, in the genetic modification, received genetic material which imparted particularly advantageous useful traits to these plants. Examples of such traits are better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products. Further and particularly emphasized examples of such traits are a better defence of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), maize, soya beans, potatoes, cotton, tobacco, oilseed rape and also fruit plants (with the fruits apples, pears, citrus fruits and grapes), and particular emphasis is given to maize, soya beans, potatoes, cotton, tobacco and oilseed rape. Traits that are emphasized are in particular increased defence of the plants against insects, arachnids, nematodes and worms by toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF and also combinations thereof) (hereinbelow referred to as “Bt plants”). Traits that are also particularly emphasized are the increased defence of the plants against fungi, bacteria and viruses by systemic acquired resistance (SAR), systemin, phytoalexius, elicitors and resistance genes and correspondingly expressed proteins and toxins. Traits that are furthermore particularly emphasized are the increased tolerance of the plants to certain herbicidally active compounds, for example imidazolinones, sulphonylureas, glyphosate or phosphinotricin (for example the “PAT” gene). The genes which impart the desired traits in question can also be present in combination with one another in the transgenic plants. Examples of “Bt plants” which may be mentioned are maize varieties, cotton varieties, soya bean varieties and potato varieties which are sold under the trade names YIELD GARD® for example maize, cotton, soya beans), KnockOut® (for example maize), StarLink® (for example maize), Bollgard® (cotton), Nucotn® (cotton) and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya bean), Liberty Link® (tolerance to phosphinotricin, for example oilseed rape), IMI® (tolerance to imidazolinones) and STS® (tolerance to sulphonylureas, for example maize). Herbicide-resistant plants (plants bred in a conventional manner for herbicide tolerance) which may be mentioned include the varieties sold under the name Clearfield® (for example maize). Of course, these statements also apply to plant cultivars having these genetic traits or genetic traits still to be developed, which plants will be developed and/or marketed in the future.

The plants listed can be treated according to the invention in a particularly advantageous manner with the compounds of the general formula I and/or the active compound mixtures according to the invention. The preferred ranges stated above for the active compounds or mixtures also apply to the treatment of these plants. Particular emphasis is given to the treatment of plants with the compounds or mixtures specifically mentioned in the present text.

The active compounds according to the invention act not only against plant, hygiene and stored product pests, but also in the veterinary medicine sector against animal parasites (ectoparasites), such as hard ticks, soft ticks, mange mites, leaf mites, flies (biting and licking), parasitic fly larvae, lice, hair lice, feather lice and fleas. These parasites include:

From the order of the Anoplurida, for example, Haematopinus spp., Linognathus spp., Pediculus spp., Phtirus spp. and Solenopotes spp.
From the order of the Mallophagida and the suborders Amblycerina and Ischnocerina, for example, Trimenopon spp., Menopon spp., Trinoton spp., Bovicola spp., Werneckiella spp., Lepikentron spp., Damalina spp., Trichodectes spp. and Felicola spp.
From the order of the Diptera and the suborders Nematocerina and Brachycerina, for example, Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp. and Melophagus spp.
From the order of the Siphonapterida, for example Pulex spp., Ctenocephalides spp., Xenopsylla spp. and Ceratophyllus spp.
From the order of the Heteropterida, for example, Cimex spp., Triatoma spp., Rhodnius spp. and Panstrongylus spp.
From the order of the Blattarida, for example Blatta orientalis, Periplaneta americana, Blattella germanica and Supella spp.
From the subclass of the Acaria (Acarida) and the orders of the Meta- and Mesostigmata, for example, Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Stemostoma spp, and Varroa spp.
From the order of the Actinedida (Prostigmata) and Acaridida (Astigmata), for example, Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp. and Laminosioptes spp.

They have, for example, excellent activity against the development stages of ticks, such as, for example, Boophilus microplus, and against parasitic flies, such as, for example, Lucilia cuprina and against fleas, such as, for example, Ctenocephalides felis.

The active compounds of the formula (I) according to the invention are also suitable for controlling arthropods which infest agricultural productive livestock, such as, for example, cattle, sheep, goats, horses, pigs, donkeys, camels, buffalo, rabbits, chickens, turkeys, ducks, geese and bees, other pets, such as, for example, dogs, cats, caged birds and aquarium fish, and also so-called test animals, such as, for example, hamsters, guinea pigs, rats and mice. By controlling these arthropods, cases of death and reduction in productivity (for meat, milk, wool, hides, eggs, honey etc.) should be diminished, so that more economic and easier animal husbandry is possible by use of the active compounds according to the invention.

The active compounds according to the invention are used in the veterinary sector in a known manner by enteral administration in the form of, for example, tablets, capsules, potions, drenches, granules, pastes, boluses, the feed-through process and suppositories, by parenteral administration, such as, for example, by injection (intramuscular, subcutaneous, intravenous, intraperitoneal and the like), implants by nasal administration, by dermal use in the form, for example, of dipping or bathing, spraying, pouring on and spotting on, washing and powdering, and also with the aid of moulded articles containing the active compound, such as collars, ear marks, tail marks, limb bands, halters, marking devices and the like.

When used for cattle, poultry, pets and the like, the active compounds of the formula (I) can be used as formulations (for example powders, emulsions, free-flowing compositions), which comprise the active compounds in an amount of 1 to 80% by weight, directly or after 100 to 10 000-fold dilution, or they can be used as a chemical bath.

It has furthermore been found that the compounds according to the invention also have a strong insecticidal action against insects which destroy industrial materials.

The following insects may be mentioned as examples and as preferred—but without a limitation:

Beetles, such as Hylotrupes bajulus, Chlorophorus pilosis, Anobium punctatum, Xestobium rufovillosum, Ptilinus pecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthes rugicollis, Xyleborus spec., Tryptodendron spec., Apate monachus, Bostrychus capucins, Heterobostrychus brunneus, Sinoxylon spec., Dinoderus minutes;
Hymenopterons, such as Sirex juvencus, Urocerus gigas, Urocerus gigas taignus, Urocerus augur;
Termites, such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis, Coptotermes formosanus;
Bristletails, such as Lepisma saccharine

Industrial materials in the present connection are to be understood as meaning non-living materials, such as, preferably, plastics, adhesives, sizes, papers and cards, leather, wood and processed wood products and coating compositions.

Wood and processed wood products are materials to be protected, especially preferably, from insect infestation.

Wood and processed wood products which can be protected by the agents according to the invention or mixtures comprising these are to be understood as meaning, for example:

Building timber, wooden beams, railway sleepers, bridge components, boat jetties, wooden vehicles, boxes, pallets, containers, telegraph poles, wood panelling, wooden windows and doors, plywood, chipboard, joinery or wooden products which are used quite generally in house-building or in building joinery.

The active compounds can be used as such, in the form of concentrates or in generally customary formulations, such as powders, granules, solutions, suspensions, emulsions or pastes.

The formulations mentioned can be prepared in a manner known per se, for example by mixing the active compounds with at least one solvent or diluent, emulsifier, dispersing agent and/or binder or fixing agent, a water repellent, if appropriate siccatives and UV stabilizers and if appropriate dyestuffs and pigments, and also other processing auxiliaries.

The insecticidal compositions or concentrates used for the preservation of wood and wood-derived timber products comprise the active compound according to the invention in a concentration of 0.0001 to 95% by weight, in particular 0.001 to 60% by weight.

The amount of the compositions or concentrates employed depends on the nature and occurrence of the insects and on the medium. The optimum amount employed can be determined for the use in each case by a series of tests. In general, however, it is sufficient to employ 0.0001 to 20% by weight, preferably 0.001 to 10% by weight, of the active compound, based on the material to be preserved.

Solvents and/or diluents which are used are an organic chemical solvent or solvent mixture and/or an oily or oil-like organic chemical solvent or solvent mixture of low volatility and/or a polar organic chemical solvent or solvent mixture and/or water, and if appropriate an emulsifier and/or wetting agent.

Organic chemical solvents which are preferably used are oily or oil-like solvents having an evaporation number above 35 and a flashpoint above 30° C., preferably above 45° C. Substances which are used as such oily or oil-like water-insoluble solvents of low volatility are appropriate mineral oils or aromatic fractions thereof, or solvent mixtures containing mineral oils, preferably white spirit, petroleum and/or alkylbenzene.

Mineral oils having a boiling range from 170 to 220° C., white spirit having a boiling range from 170 to 220° C., spindle oil having a boiling range from 250 to 3.50° C., petroleum and aromatics having a boiling range from 160 to 280° C., turpentine oil and the like, are advantageously employed.

In a preferred embodiment, liquid aliphatic hydrocarbons having a boiling range from 180 to 210° C. or high-boiling mixtures of aromatic and aliphatic hydrocarbons having a boiling range from 180 to 220° C. and/or spindle oil and/or monochloronaphthalene, preferably α-monochloronaphthalene, are used.

The organic oily or oil-like solvents of low volatility which have an evaporation number above 35 and a flashpoint above 30° C., preferably above 45° C., can be replaced in part by organic chemical solvents of high or medium volatility, provided that the solvent mixture likewise has an evaporation number above 35 and a flashpoint above 30° C., preferably above 45° C., and that the insecticide/fungicide mixture is soluble or emulsifiable in this solvent mixture.

According to a preferred embodiment, some of the organic chemical solvent or solvent mixture is replaced by an aliphatic polar organic chemical solvent or solvent mixture. Aliphatic organic chemical solvents containing hydroxyl and/or ester and/or ether groups, such as, for example, glycol ethers, esters or the like, are preferably used.

Organic chemical binders which are used in the context of the present invention are the synthetic resins and/or binding drying oils which are known per se, are water-dilutable and/or are soluble or dispersible or emulsifiable in the organic chemical solvents employed, in particular binders consisting of or comprising an acrylate resin, a vinyl resin, for example polyvinyl acetate, polyester resin, polycondensation or polyaddition resin, polyurethane resin, alkyd resin or modified alkyd resin, phenolic resin, hydrocarbon resin, such as indene-cumarone resin, silicone resin, drying vegetable oils and/or drying oils and/or physically drying binders based on a natural and/or synthetic resin.

The synthetic resin used as the binder can be employed in the form of an emulsion, dispersion or solution. Bitumen or bituminous substances can also be used as binders in an amount of up to 10% by weight. Dyestuffs, pigments, water-repelling agents, odour correctants and inhibitors or anticorrosive agents and the like which are known per se can additionally be employed.

It is preferred according to the invention for the composition or concentrate to comprise, as the organic chemical binder, at least one alkyd resin or modified alkyd resin and/or a drying vegetable oil. Alkyd resins having an oil content of more than 45% by weight, preferably 50 to 68% by weight, are preferably used according to the invention.

All or some of the binder mentioned can be replaced by a fixing agent (mixture) or a plasticizer (mixture). These additives are intended to prevent evaporation of the active compounds and crystallization or precipitation. They preferably replace 0.01 to 30% of the binder (based on 100% of the binder employed).

The plasticizers originate from the chemical classes of phthalic acid esters, such as dibutyl, dioctyl or benzyl butyl phthalate, phosphoric acid esters, such as tributyl phosphate, adipic acid esters, such as di-(2-ethylhexyl)adipate, stearates, such as butyl stearate or amyl stearate, oleates, such as butyl oleate, glycerol ethers or higher molecular weight glycol ethers, glycerol esters and p-toluenesulphonic acid esters.

Fixing agents are based chemically on polyvinyl alkyl ethers, such as, for example, polyvinyl methyl ether or ketones, such as benzophenone or ethylenebenzophenone.

Possible solvents or diluents are, in particular, also water, if appropriate as a mixture with one or more of the abovementioned organic chemical solvents or diluents, emulsifiers and dispersing agents.

Particularly effective preservation of wood is achieved by impregnation processes on a large industrial scale, for example vacuum, double vacuum or pressure processes.

The ready-to-use compositions can also comprise other insecticides, if appropriate, and also one or more fungicides, if appropriate.

Possible additional mixing partners are, preferably, the insecticides and fungicides mentioned in WO 94/29 268. The compounds mentioned in this document are an explicit constituent of the present application.

Especially preferred mixing partners which may be mentioned are insecticides, such as chlorpyriphos, phoxim, silafluofin, alphamethrin, cyfluthrin, cypermethrin, deltamethrin, permethrin, imidacloprid, NI-25, flufenoxuron, hexaflumuron, transfluthrin, thiacloprid, methoxyfenozide, triflumuron, clothianidin, spinosad, tefluthrin,

and also fungicides, such as epoxyconazole, hekaconazole, azaconazole, propiconazole, tebuconazole, cyproconazole, metconazole, imazalil, dichlorfluanid, tolylfluanid, 3-iodo-2-propinyl-butyl carbamate, N-octyl-isothiazolin-3-one and 4,5-dichloro-N-octylisothiazolin-3-one.

The compounds according to the invention can at the same time be employed for protecting objects which come into contact with saltwater or brackish water, such as hulls, screens, nets, buildings, moorings and signalling systems, against fouling.

Fouling by sessile Oligochaeta, such as Serpulidae, and by shells and species from the Ledamorpha group (goose barnacles), such as various Lepas and Scalpellum species, or by species from the Balanomorpha group (acorn barnacles), such as Balanus or Pollicipes species, increases the frictional drag of ships and, as a consequence, leads to a marked increase in operational costs owing to higher energy consumption and additionally frequent residence in the dry dock.

Apart from fouling by algae, for example Ectocarpus sp. and Ceramium sp., fouling by sessile Entomostraka groups, which come under the generic term Cirripedia (cirriped crustaceans), is of particular importance.

Surprisingly, it has now been found that the compounds according to the invention, alone or in combination with other active compounds, have an outstanding antifouling action.

Using the compounds according to the invention, alone or in combination with other active compounds, allows the use of heavy metals such as, for example, in bis(trialkyltin) sulphides, tri-n-butyltin laurate, tri-n-butyltin chloride, copper(I) oxide, triethyltin chloride, tri-n-butyl(2-phenyl-4-chlorophenoxy)tin, tributyltin oxide, molybdenum disulphide, antimony oxide, polymeric butyl titanate, phenyl-(bispyridine)-bismuth chloride, tri-n-butyltin fluoride, manganese ethylenebisthiocarbamate, zinc dimethyldithiocarbamate, zinc ethylenebisthiocarbamate, zinc salts and copper salts of 2-pyridinethiol 1-oxide, bisdimethyldithiocarbamoylzine ethylene-bisthiocarbamate, zinc oxide, copper(I) ethylene-bisdithiocarbamate, copper thiocyanate, copper naphthenate and tributyltin halides to be dispensed with, or the concentration of these compounds to be substantially reduced.

If appropriate, the ready-to-use antifouling paints can additionally comprise other active compounds, preferably algicides, fungicides, herbicides, molluscicides, or other antifouling active compounds.

Preferably suitable components in combinations with the antifouling compositions according to the invention are:

algicides such as 2-tert-butylamino-4-cyclopropylamino-6-methylthio-1,3,5-triazine, dichlorophen, diuron, endothal, fentin acetate, isoproturon, methabenzthiazuron, oxyfluorfen, quinoclamine and terbutryn; fungicides such as benzo[b]thiophenecarboxylic acid cyclohexylamide S,S-dioxide, dichlofluanid, fluorfolpet, 3-iodo-2-propinyl butylcarbamate, tolylfluanid and azoles such as azaconazole, cyproconazole, epoxyconazole, hexaconazole, metconazole, propiconazole and tebuconazole; molluscicides such as fentin acetate, metaldehyde, methiocarb, niclosamid, thiodicarb and trimethacarb; or conventional antifouling active compounds such as 4,5-dichloro-2-octyl-4-isothiazolin-3-one, diiodomethylparatryl sulphone, 2-(N,N-dimethylthiocarbamoylthio)-5-nitrothiazyl, potassium, copper, sodium and zinc salts of 2-pyridinethiol 1-oxide, pyridine-triphenylborane, tetrabutyldistannoxane, 2,3,5,6-tetrachloro-4-(methylsulphonyl)-pyridine, 2,4,5,6-tetrachloroisophthalonitrile, tetramethylthiuram disulphide and 2,4,6-trichlorophenylmaleiimide.

The antifouling compositions used comprise the active compound according to the invention of the compositions according to the invention in a concentration of 0.001 to 50% by weight, in particular 0.01 to 20% by weight.

Moreover, the antifouling compositions according to the invention comprise the customary components such as, for example, those described in Ungerer, Chem. Ind. 1985, 37, 730-732 and Williams, Antifouling Marine Coatings, Noyes, Park Ridge, 1973.

Besides the algicidal, fungicidal, molluscicidal active compounds and insecticidal active compounds according to the invention, antifouling paints comprise, in particular, binders.

Examples of recognized binders are polyvinyl chloride in a solvent system, chlorinated rubber in a solvent system, acrylic resins in a solvent system, in particular in an aqueous system, vinyl chloride/vinyl acetate copolymer systems in the form of aqueous dispersions or in the form of organic solvent systems, butadiene/styrene/acrylonitrile rubbers, drying oils such as linseed oil, resin esters or modified hardened resins in combination with tar or bitumens, asphalt and epoxy compounds, small amounts of chlorine rubber, chlorinated polypropylene and vinyl resins.

If appropriate, paints also comprise inorganic pigments, organic pigments or colorants which are preferably insoluble in salt water. Paints may furthermore comprise materials such as colophonium to allow controlled release of the active compounds. Furthermore, the paints may comprise plasticizers, modifiers which affect the rheological properties and other conventional constituents. The compounds according to the invention or the abovementioned mixtures may also be incorporated into self-polishing antifouling systems.

The active compounds according to the invention are also suitable for controlling animal pests, in particular insects, arachnids and mites, which are found in enclosed spaces such as, for example, dwellings, factory halls, offices, vehicle cabins and the like. They can be employed alone or in combination with other active compounds and auxiliaries in domestic insecticide products for controlling these pests. They are active against sensitive and resistant species and against all developmental stages. These pests include:

From the order of the Scorpionidea, for example, Buthus occitanus.
From the order of the Acarina, for example, Argas persicus, Argas reflexus, Bryobia spp., Dermanyssus gallinae, Glyciphagus domesticus, Ornithodorus moubat, Rhipicephalus sanguineus, Trombicula alfreddugesi, Neutrombicula autumnalis, Dermatophagoides pteronissimus, Dermatophagoides forinae.
From the order of the Araneae, for example, Aviculariidae, Araneidae.
From the order of the Opiliones, for example, Pseudoscorpiones chelifer, Pseudoscorpiones cheiridium, Opiliones phalangium.
From the order of the Isopoda, for example, Oniscus asellus, Porcellio scaber.
From the order of the Diplopoda, for example, Blaniulus guttulatus, Polydesmus spp.
From the order of the Chilopoda, for example, Geophilus spp.
From the order of the Zygentoma, for example, Ctenolepisma spp., Lepisma saccharina, Lepismodes inquilinus.
From the order of the Blattaria, for example, Blatta orientalies, Blattella germanica, Blattella asahinai, Leucophaea maderae, Panchlora spp., Parcoblatta spp., Periplaneta australasiae, Periplaneta americana, Periplaneta brunnea, Periplaneta fuliginosa, Supella longipalpa.
From the order of the Saltatoria, for example, Acheta domesticus.
From the order of the Dermaptera, for example, Forficula auricularia.
From the order of the Isoptera, for example, Kalotermes spp., Reticulitermes spp.
From the order of the Psocoptera, for example, Lepinatus spp., Liposcelis spp.
From the order of the Coleptera, for example, Anthrenus spp., Attagenus spp., Dermestes spp., Latheticus oryzae, Necrobia spp., Ptinus spp., Rhizopertha dominica, Sitophilus granarius, Sitophilus oryzae, Sitophilus zeamais, Stegobium paniceum.
From the order of the Diptera, for example, Aedes aegypti, Aedes albopictus, Aedes taeniorhynchus, Anopheles spp., Calliphora erythrocephala, Chrysozona pluvialis, Culex quinquefasciatus, Culex pipiens, Culex tarsalis, Drosophila spp., Fannia canicuaris, Musca domestica, Phlebotomus spp., Sarcophaga carnaria, Simulium spp., Stomoxys calcitrans, Tipula paludosa.
From the order of the Lepidoptera, for example, Achroia grisella, Galleria mellonella, Plodia interpunctella, Tinea cloacella, Tinea pellionella, Tineola bisselliella.
From the order of the Siphonaptera, for example, Ctenocephalides canis, Ctenocephalides felis, Pulex irritans, Tunga penetrans, Xenopsylla cheopis.
From the order of the Hymenoptera, for example, Camponotus herculeanus, Lasius fuliginosus, Lasius niger, Lasius umbratus, Monomorium pharaonis, Paravespula spp., Tetramorium caespitum.
From the order of the Anoplura, for example, Pediculus humanus capitis, Pediculus humanus corporis, Phthirus pubis.
From the order of the Heteroptera, for example, Cimex hemipterus, Cimex lectularius, Rhodinus prolixus, Triatoma infestans.

In the field of household insecticides, they are used alone or in combination with other suitable active compounds, such as phosphoric acid esters, carbamates, pyrethroids, growth regulators or active compounds from other known classes of insecticides.

They are used as aerosols, pressure-free spray products, for example pump and atomizer sprays, automatic fogging systems, foggers, foams, gels, evaporator products with evaporator tablets made of cellulose or polymer, liquid evaporators, gel and membrane evaporators, propeller-driven evaporators, energy-free, or passive, evaporation systems, moth papers, moth bags and moth gels, as granules or dusts, in baits for spreading or in bait stations.

The preparation and use of the substances according to the invention are illustrated by the examples below.

### PREPARATION EXAMPLES

### Example 1

### Synthesis of 4-(1-difluoromethyl-2,2,2-trifluoro-ethyl)-2-methyl-phenylamine

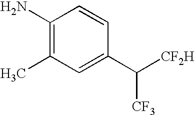

### Step 1: 4-(1-Difluoromethyl-1,2,2,2-tetrafluoro-ethyl)-2-methyl-phenylamine

14.0 g (41.66 mmol) 4-[1-(bromo-difluoro-methyl)-1,2,2,2-tetrafluoro-ethyl]-2-methyl-phenylamine was solved in 250 ml toluene, 0.68 g (4.17 mmol) α,α′-azo-bis-isobutyronitrile and 18.2 g (62.49 mmol) tributyl-stannane were added and the mixture was refluxed for 30 minutes. The solvent was removed under reduced pressure and the residue solved in tert-butyl-methyl ether. A concentrated solution of potassium fluoride was added and the mixture was stirred for 2 hours. The organic layer was separated, washed with a saturated sodium chloride solution, dried with sodium sulphate and concentrated in vacuo. The crude residue was further purified by column chromatography with dichloromethane as solvent to obtain the product as a yellow oil.

Yield: 7.8 g (69.2%)

HPLC: log P (pH 2.3)=2.74

### Step 2: 4-(1-Difluoromethyl-2,2,2-trifluoro-ethyl)-2-methyl-phenylamine

3.1 g (12.05 mmol) 4-(1-difluoromethyl-1,2,2,2-tetrafluoro-ethyl)-2-methyl-phenylamine was added under argon to a mixture of 0.547 g (14.47 mmol) sodium borohydride in 10 ml dimethyl sulphoxide and stirred at 50° C. for 30 minutes. Water was added and the mixture was extracted twice with tert-butyl-methyl ether. The combined organic layers were washed with water and a saturated sodium chloride solution, dried with sodium sulphate and the solvent was removed in vacuo to obtain a yellow oil.

Yield: 1.2 g (41.6%)

HPLC: log P (pH 2.3)=2.02

### Synthesis of N¹-[4-(2-bromo-1,2,2-trifluoro-1-trifluoromethyl-ethyl)-2-methyl-phenyl]-3-iodo-N²-[2-methanesulfonyl-(S)-1-methyl-ethyl]-phthalamide

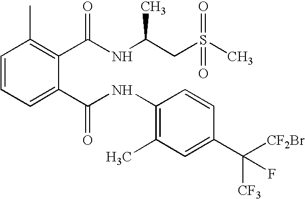

### Step 1: 3-Iodo-N-(1-methyl-2-methylsulfanyl-ethyl)-phthalamic Acid

34.73 g (126.74 mmol) 3-iodo-phthalic acid anhydride was solved in N,N-dimethyl-acetamide and at 10° C. a solution of 16.0 g (152.10 mmol) (S)-1-methyl-2-methylsulfanyl-ethylamine in N,N-dimethyl-acetamide was added over 60 minutes. The mixture was stirred for further 60 minutes, a solution of 16.5 g (164.76 mmol) sodium hydroxide in water was added over 70 minutes and stirred for 12 hours. The solvent was removed under reduced pressure and the residue diluted with water. tert-Butyl-methyl ether was added to the solution and the mixture was acidified with hydrochloric acid until pH=1-2. The organic layer was separated, washed with water and a saturated sodium chloride solution and dried with sodium sulphate. After removing of the solvent in vacuo the product was obtained in form of an orange oil which crystallizes within a few hours.

Yield: 22.3 g (46.4%)

Mp.: 132-134° C.

### Step 2: 4-Iodo-3-[(S)-1-methyl-2-methylsulfanyl-ethylimino]-3H-isobenzofuran-1-one

15.1 g (38.82 mmol) 3-iodo-N-[(S)-1-methyl-2-methylsulfanyl-ethyl]-phthalamic acid was solved in dichloromethane and 6.02 g (71.67 mmol) sodium hydrogen carbonate in water was added at 40° C. and at the same temperature 5.64 g (59.73 mmol) methyl chloroformate over 15 minutes. The mixture was stirred at 50° C. for 1 hour and diluted with water. The organic layer was separated and extracted two times with dichloromethane. The combined organic phases were washed with water, dried with sodium sulphate and the solvent was removed in vacuo. The yellow oil crystallizes within a few hours.

Yield: 10.5 g (69.4%)

HPLC: log P (pH 2.3)=3.87

### Step 3: N¹-[4-(2-Bromo-1,2,2-trifluoro-1-trifluoromethyl-ethyl)-2-methyl-phenyl]-3-iodo-N²-[(S)-1-methyl-2-methylsulfanyl-ethyl]-phthalamide (Compound 9 in Table 1)

845.9 mg (2.52 mmol) 4-[1-(bromo-difluoro-methyl)-1,2,2,2-tetrafluoro-ethyl]-2-methyl-phenyl-amine was solved in dichloroethane, catalytic amounts of concentrated hydrochloric acid were added and the mixture was heated to 55° C. At this temperature a solution of 1.0 g (2.77 mmol) 4-iodo-3-[(S)-1-methyl-2-methylsulfanyl-ethylimino]-3H-isobenzofuran-1-one was added and the mixture was stirred for 30 minutes at 65° C. The solvent was removed in vacuo and the crude residue was further purified by column chromatography with dichloromethane as solvent to obtain a white solid.

Yield: 970 mg (51.4%)

Mp.: 124° C.

### Step 4: N¹-[4-(2-Bromo-1,2,2-trifluoro-1-trifluoromethyl-ethyl)-2-methyl-phenyl]-3-iodo-N²-[2-methanesulfinyl-(S)-1-methyl-ethyl]-phthalamide (Compound 65 in Table 1)

200.0 mg (0.287 mmol) N¹-[4-(2-bromo-1,2,2-trifluoro-1-trifluoromethyl-ethyl)-2-methyl-phenyl]-3-iodo-N²-[(S)-1-methyl-2-methylsulfanyl-ethyl]-phthalamide was dissolved in dichloroethane and 2.64 mg (0.057 mmol) formic acid and 39.03 mg (0.344 mmol) hydrogen peroxide were added at 60° C. The mixture was stirred at 60° C. for 30 minutes. At 50° C. 15 ml of a sodium hydrogen sulfite solution (10%) were added and the mixture stirred for 10 minutes. The organic layer was separated, dried with sodium sulphate and the solvent removed under reduced pressure. The crude product was further purified by column chromatography with ethylacetate/tert-butyl-methyl-ether/methanol (80:15:5) as solvent.

Yield: 150 mg (73.3%)

Mp.: 120° C.

### Step 5: N¹-[4-(2-Bromo-1,2,2-trifluoro-1-trifluoromethyl-ethyl)-2-methyl-phenyl]-3-iodo-N²-[2-methanesulfonyl-(S)-1-methyl-ethyl]-phthalamide (Compound 19 in Table 1)

400.0 mg (0.574 mmol) N¹-[4-(2-Bromo-1,2,2-trifluoro-1-trifluoromethyl-ethyl)-2-methyl-phenyl]-3-iodo-N²-[2-methanesulfinyl-(S)-1-methyl-ethyl]-phthalamide was dissolved in dichloroethane and 26.4 mg (0.574 mmol) formic acid, 11.25 mg (0.115 mmol) sulphuric acid and 126.6 mg (1.434 mmol) hydrogen peroxide were added at 60° C. The mixture was stirred at 60° C. for 30 minutes. At 50° C. 15 ml of a sodium hydrogen sulphite solution (10%) were added and stirred for 10 minutes. The organic layer was separated, dried with sodium sulphate and the solvent removed under reduced pressure. The crude product was further purified by column chromatography with cyclohexan/ethylacetate 2:1.

Yield: 270 mg (62.0%)

Mp.: 128° C.

The following compounds of the formula (I) are obtained analogously to the above examples and the general description.

TABLE 1

| | |
|---|---|
| | (I) |
| 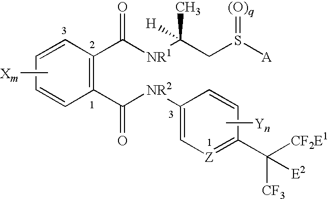 | |

| No. | A | q | R¹ | R² | E¹ | E² | X | Y | Z | m.p./° C. |
|---|---|---|---|---|---|---|---|---|---|---|
| 1 | Et | 0 | H | H | Br | F | 3-I | 2-Me | CH | 114 |
| 2 | Et | 2 | H | H | Br | F | 3-I | 2-Me | CH | 133 |
| 3 | Et | 0 | H | H | H | F | 3-I | 2-Cl | CH | 187 |
| 4 | Et | 0 | H | H | H | H | 3-I | 2-Me | CH | 105 |
| 5 | Et | 0 | H | H | H | F | 3-I | 2-Me | CH | 105 |
| 6 | Et | 0 | H | H | H | F | 3-I | 2-Br | CH | 183 |
| 7 | Et | 2 | H | H | H | H | 3-I | 2-Me | CH | 115 |
| 8 | Et | 0 | H | H | H | F | 3-I | 2-F | CH | 138-141 |
| 9 | Me | 0 | H | H | Br | F | 3-I | 2-Me | CH | 124 |
| 10 | Me | 0 | H | H | H | F | 3-I | 2-Me | CH | 112 |
| 11 | Me | 0 | H | H | H | H | 3-I | 2-Me | CH | 184 |
| 12 | Me | 0 | H | H | H | F | 3-I | 2-Cl | CH | 193 |
| 13 | Et | 2 | H | H | H | F | 3-I | 2-Me | CH | 112 |
| 14 | Me | 0 | H | H | H | H | 3-I | 2-Cl | CH | 175 |
| 15 | Me | 0 | H | H | H | F | 3-I | 2-Br | CH | 181 |
| 16 | Me | 0 | H | H | H | F | 3-I | 2-F | CH | 130 |
| 17 | Me | 0 | H | H | H | H | 3-I | 2-F | CH | 102 |
| 18 | Me | 2 | H | H | H | F | 3-I | 2-Me | CH | 124 |
| 19 | Me | 2 | H | H | Br | F | 3-I | 2-Me | CH | 128 |
| 20 | Me | 2 | H | H | H | H | 3-I | 2-Me | CH | 123 |
| 21 | Et | 0 | H | H | H | H | 3-I | 2-F | CH | 144 |
| 22 | Me | 0 | H | H | H | H | 3-I | 2-Br | CH | 167 |
| 23 | Et | 0 | H | H | H | H | 3-I | 2-Cl | CH | 165 |
| 24 | Et | 0 | H | H | H | H | 3-I | 2-Br | CH | 160 |
| 25 | Et | 2 | H | H | H | F | 3-I | 2-Cl | CH | 132-135 |
| 26 | Et | 2 | H | H | H | H | 3-I | 2-Cl | CH | 115-118 |
| 27 | Me | 2 | H | H | H | F | 3-I | 2-Cl | CH | 184 |
| 28 | Me | 2 | H | H | H | H | 3-I | 2-Cl | CH | 116-120 |
| 29 | Me | 0 | H | H | H | F | 3-I | 2-CN | CH | 147-149 |
| 30 | Me | 2 | H | H | H | F | 3-I | 2-Br | CH | 112-114 |
| 31 | Me | 2 | H | H | H | H | 3-I | 2-Br | CH | 198 |
| 32 | Me | 2 | H | H | H | F | 3-I | 2-F | CH | 124-126 |
| 33 | Me | 2 | H | H | H | H | 3-I | 2-F | CH | 144-147 |
| 34 | Et | 2 | H | H | H | F | 3-I | 2-Br | CH | 134 |
| 35 | Et | 2 | H | H | H | H | 3-I | 2-Br | CH | 122 |
| 36 | Et | 2 | H | H | H | F | 3-I | 2-F | CH | 142-144 |
| 37 | Et | 2 | H | H | H | H | 3-I | 2-F | CH | 128 |
| 38 | Me | 0 | H | H | Br | F | 3-Cl | 2-Me | CH | 184-186 |
| 39 | Me | 0 | H | H | H | F | 3-Cl | 2-Me | CH | 183 |
| 40 | Me | 0 | H | H | H | H | 3-Cl | 2-Me | CH | 105 |
| 41 | Me | 0 | H | H | H | F | 3-Cl | 2-Cl | CH | 156 |
| 42 | Me | 0 | H | H | H | H | 3-Cl | 2-Cl | CH | 106 |
| 43 | Me | 2 | H | H | Br | F | 3-Cl | 2-Me | CH | 100-104 |
| 44 | Me | 2 | H | H | H | F | 3-Cl | 2-Me | CH | 101-103 |
| 45 | Et | 0 | H | H | Br | F | 3-Cl | 2-Me | CH | 127 |
| 46 | Me | 2 | H | H | H | H | 3-Cl | 2-Me | CH | 100-103 |
| 47 | Me | 2 | H | H | H | F | 3-Cl | 2-Cl | CH | 180 |
| 48 | Me | 2 | H | H | H | H | 3-Cl | 2-Cl | CH | 198 |
| 49 | Et | 2 | H | H | Br | F | 3-Cl | 2-Me | CH | 125 |
| 50 | Et | 0 | H | H | H | F | 3-Cl | 2-Cl | CH | 124 |
| 51 | Et | 0 | H | H | H | H | 3-Cl | 2-Cl | CH | 125 |
| 52 | Et | 0 | H | H | Br | F | 3-Br | 2-Me | CH | 115 |
| 53 | Et | 0 | H | H | H | F | 3-Br | 2-Cl | CH | 148 |
| 54 | Et | 0 | H | H | H | H | 3-Br | 2-Cl | CH | 100 |
| 55 | Et | 2 | H | H | H | F | 3-Cl | 2-Cl | CH | 175 |
| 56 | Et | 2 | H | H | H | H | 3-Cl | 2-Cl | CH | 116 |
| 57 | Et | 2 | H | H | Br | F | 3-Br | 2-Me | CH | 122 |
| 58 | Et | 2 | H | H | H | F | 3-Br | 2-Cl | CH | 118 |
| 59 | Et | 2 | H | H | H | H | 3-Br | 2-Cl | CH | 121 |
| 60 | Me | 1 | H | H | H | H | 3-I | 2-Me | CH | 98-102 |
| 61 | Et | 1 | H | H | H | H | 3-I | 2-Me | CH | 110 |
| 62 | Me | 1 | H | H | H | H | 3-Cl | 2-Me | CH | 128 |
| 63 | Me | 0 | H | H | Br | F | 3-Br | 2-Me | CH | 192 |
| 64 | Me | 2 | H | H | Br | F | 3-Br | 2-Me | CH | 122 |
| 65 | Me | 1 | H | H | Br | F | 3-I | 2-Me | CH | 120 |
| 66 | Me | 1 | H | H | H | F | 3-I | 2-Me | CH | 122 |
| 67 | Me | 0 | H | H | H | F | 3-Br | 2-Br | CH | 138-142 |
| 68 | Et | 0 | H | H | H | F | 3-Br | 2-Br | CH | 140-142 |
| 69 | Et | 2 | H | H | H | F | 3-Br | 2-Br | CH | 150-151 |

The log P values given in the preparation examples and tables above are determined in accordance with EEC Directive 79/831 Annex V.A8 by HPLC (High Performance Liquid Chromatography) on a reversed-phase column (C 18). Temperature: 43° C.

In the acidic range, determination is carried out at pH 2.3 using the mobile phases 0.1% aqueous phosphoric acid and acetonitrile; linear gradient from 10% acetonitrile to 90% acetonitrile (in the tables marked with a)).

In the neutral range, determination is carried out at pH 7.5 using the mobile phases 0.01 molar aqueous phosphate buffer solution and acetonitrile; linear gradient from 10% acetonitrile to 90% acetonitrile (in the tables marked with b)).

Calibration is carried out using unbranched alkan-2-ones (of 3 to 16 carbon atoms) with known lop P values (determination of the log P values by the retention times using linear interpolation between two successive alkanones).

The lambda max values were determined in the maxima of the chromatographic signals using the UV spectra from 200 nm to 400 nm.

### Use Examples

### Example A

### Test with Resistant Monoxenic Cattle Tick/SP-Resistant Parkhurst Strain Injection Method

Boophilus microplus (INJ)

| | |
|---|---|
| Test animals: | Adult satiated females of Boophilus microplus (SP-resistant |
| | Parkhurst strain) |
| Solvent: | Dimethyl sulphoxide |

10 mg active compound are dissolve in 0.5 ml Dimethylsulfoxid. Serial dilutions are made to obtain the desired rates.

The test is carried out in 5 replications. 1 μl of the solutions is injected into the abdomen, and the animals are transferred to dishes and kept in a climate-controlled room. After 7 days, activity is checked by examination for deposition of fertile eggs. Eggs whose fertility is not externally visible are stored in glass tubes in a controlled-environment cabinet until the larvae have hatched after ca. 24 days. An activity of 100% denotes that none of the ticks has laid fertile eggs.

In this test, for example, the following compounds of the preparation examples show good activity:

TABLE A
Animal parasites
Boophilus test (INJ)

| | | Concentration of active | Kill rate in % |
|---|---|---|---|
| No. | Active compounds | compound in μM | after 7 d |
| 5 | 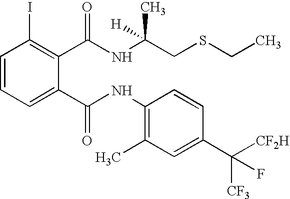 | 20 | 100 |
| 4 | 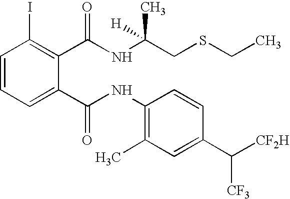 | 20 | 100 |
| 23 | 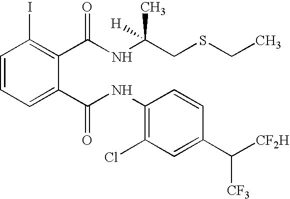 | 20 | 100 |
| 24 | 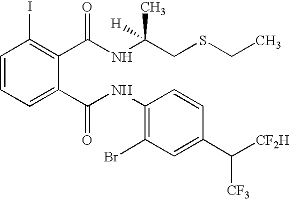 | 20 | 100 |
| 33 | 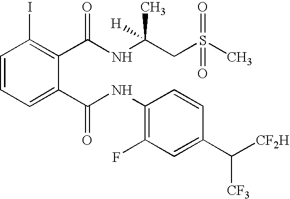 | 20 | 100 |

### Example B

### Heliothis armigera Test

| | |
|---|---|
| Solvent: | 7 parts by weight of dimethylformamide |
| Emulsifier: | 2 parts by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Soybean shoots (Glycine max) are treated by being dipped into the preparation of active compound of the desired concentration and are populated with caterpillars of Heliothis armigera while the leaves are still moist.

After the desired period of time, the kill in % is determined. 100% means that all caterpillars have been killed; 0% means that none of the caterpillars have been killed.

In this test, for example, the following compounds of the preparation examples show good activity:

TABLE B
Plant-damaging insects
Heliothis armigera test

| | | Concentration of active | Kill rate in % |
|---|---|---|---|
| No. | Active compounds | compound in ppm | after 7 d |
| 9 | 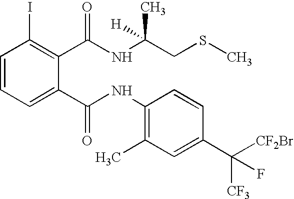 | 20 | 100 |
| 14 | 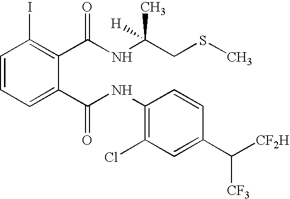 | 20 | 100 |
| 20 | 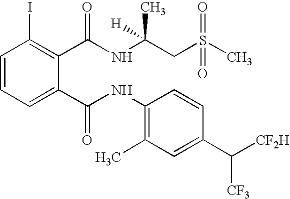 | 20 | 100 |
| 40 | 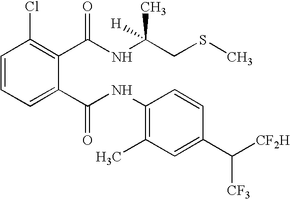 | 20 | 100 |
| 48 | 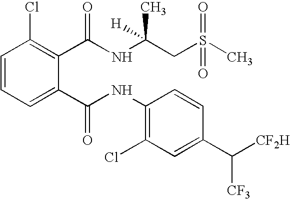 | 20 | 100 |
| 53 | 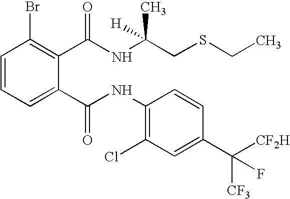 | 20 | 100 |
| 55 | 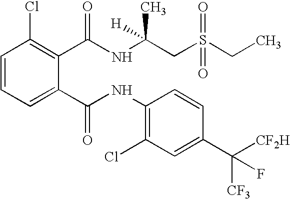 | 20 | 100 |
| 58 | 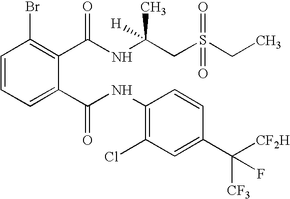 | 20 | 100 |
| 68 | 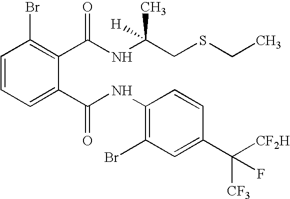 | 20 | 100 |

### Example C

### Blowfly Larval Assay

Lucilia cuprina (48 h)

| | |
|---|---|
| Species: | Lucilia cuprina 1^{st} instar larvae (age 24 hrs) |
| Solvent: | Dimethyl sulphoxide |

10 mg active compound are dissolve in 0.5 ml dimethyl sulphoxide. Serial dilutions are made to obtain the desired rates.

Approximately 20 Lucilia cuprina 1^{st} instar larvae are transferred into a test tube containing 1 cm³ of minced horse meat and 0.5 ml aqueous dilution of test compound. After 48 hrs percentage of larval mortality are recorded.

100% efficacy=all larvae are killed
0% efficacy=normally developed larvae after 48 hrs

In this test, for example, the following compounds of the preparation examples show good activity:

TABLE C
Animal parasites
Fly larvae test (Lucilia cuprina)

| | | Concentration of acitve | Kill rate in % |
|---|---|---|---|
| No. | Active compounds | compound in ppm | after 2 d |
| 1 | 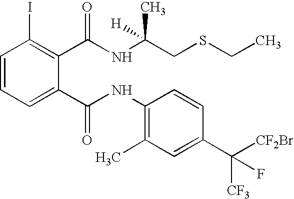 | 100 | 100 |
| 4 | 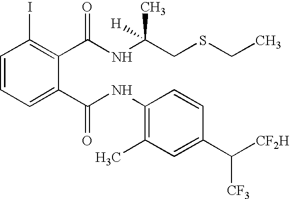 | 100 | 100 |
| 25 | 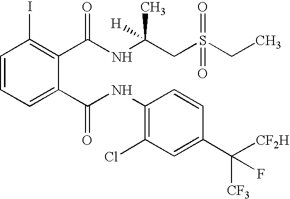 | 100 | 90 |
| 32 | 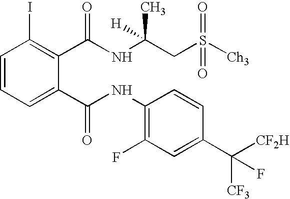 | 100 | 100 |
| 36 | 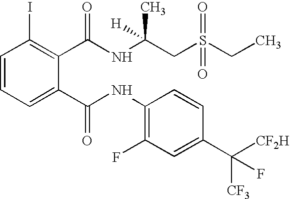 | 100 | 100 |
| 37 | 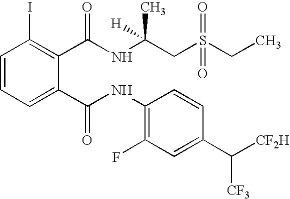 | 100 | 100 |
| 69 | 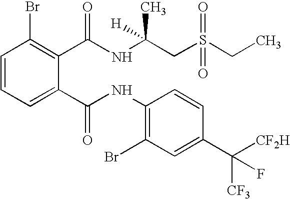 | 100 | 100 |

### Example D

### Housefly Assay

Musca domestica

| | |
|---|---|
| Species: | male and female adults (Musca domestica), fully susceptible |
| | laboratory strain (WHO(N)) |
| Solvent: | Dimethyl sulphoxide |

10 mg active compound are dissolve in 0.5 ml dimethyl sulphoxide. Serial dilutions are made to obtain the desired rates.

Prior to the assay, a piece of kitchen sponge (ca. Ø 1.5 cm) is soaked with 0.8 ml of sugar solution and 0.2 ml of aqueous compound dilution and is placed into a container (4×4×2 cm). 10 adult Musca domestica flies (WHO/N strain) are anaesthetized by CO₂ and also placed into the container. The container is closed with a perforated lid. Percentage of efficacy is monitored after 48 hrs.

100% efficacy=all flies are killed
0% efficacy=no flies are killed

In this test, for example, the following compounds of the preparation examples show good activity:

TABLE D
Animal parasites
Test with flies (Musca domestica)

| | | Concentration of active | Kill rate in % |
|---|---|---|---|
| No. | Active compounds | compound in ppm | after 4 d |
| 5 | 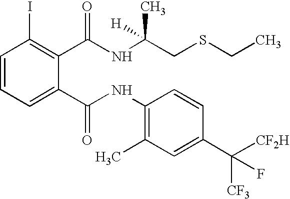 | 100 | 100 |
| 21 | 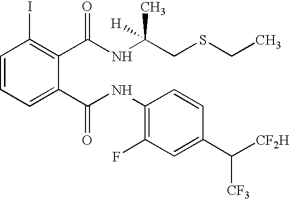 | 100 | 100 |
| 28 | 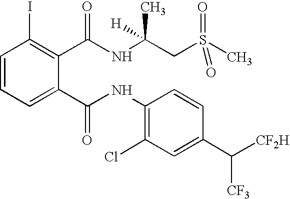 | 100 | 100 |
| 31 | 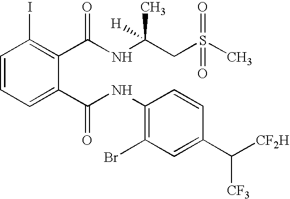 | 100 | 100 |
| 32 | 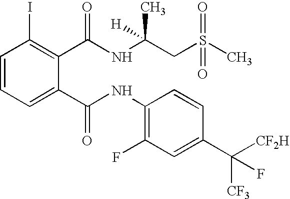 | 100 | 100 |
| 33 | 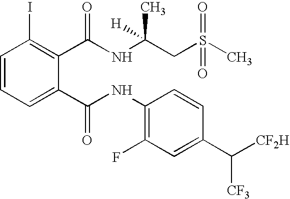 | 100 | 100 |
| 35 | 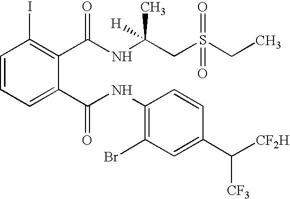 | 100 | 100 |

### Example E

### Myzus Test (Spray Application)

| | |
|---|---|
| Solvent: | 78 parts by weight of acetone |
| | 1.5 parts by weight of dimethyl formamide |
| Emulsifier: | 0.5 parts by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Chinese cabbage leaves (Brassica pekinensis), which are infested by the green peach aphid (Myzus persicae), are treated by being sprayed with the preparation of the active compound of the desired concentration.

After the specified period of time, the mortality in % is determined. 100% means that all the aphids have been killed; 0% means that none of the aphids have been killed.

In this test, for example, the following compounds of the preparation examples show good activity:

TABLE E
Plant-damaging insects
Myzus test (Spray application)

| | | Concentration of active | Kill rate in % |
|---|---|---|---|
| No. | Active compounds | compound in g/ha | after 5 d |
| 14 | 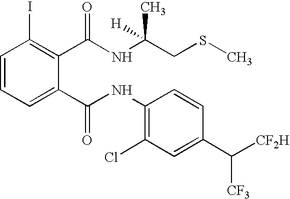 | 100 | 100 |
| 7 | 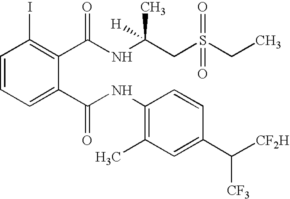 | 100 | 100 |
| 17 | 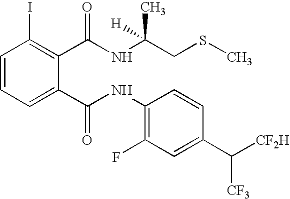 | 100 | 100 |
| 26 | 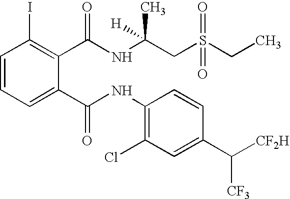 | 100 | 100 |
| 31 | 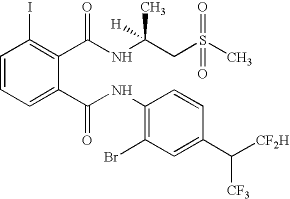 | 100 | 100 |
| 43 | 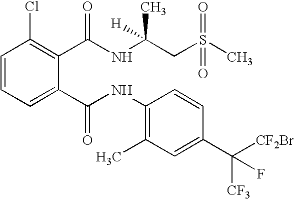 | 100 | 100 |
| 51 | 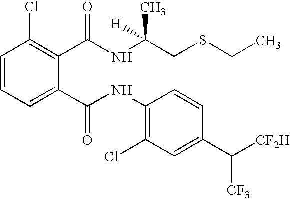 | 100 | 100 |
| 62 | 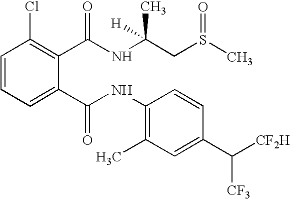 | 100 | 100 |
| 63 | 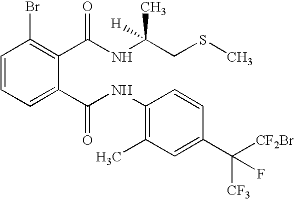 | 100 | 90 |
| 66 | 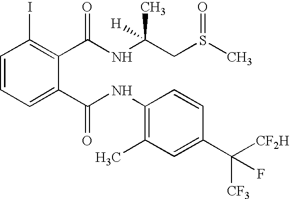 | 100 | 100 |

### Example F

### Phaedon Test (Spray Application)

| | |
|---|---|
| Solvent: | 78 parts by weight of acetone |
| | 1.5 parts by weight of dimethyl formamide |
| Emulsifier: | 0.5 parts by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Chinese cabbage leaves (Brassica pekinensis) are treated by being sprayed with the preparation of the active compound of the desired concentration and are infested with mustard beetle larvae (Phaedon cochleariae) after the leaves have dried.

After the specified period of time, the mortality in % is determined. 100% means that all the aphids have been killed; 0% means that none of the aphids have been killed.

In this test, for example, the following compounds of the preparation examples show good activity:

TABLE F
Plant-damaging insects
Phaedon test (Spray application)

| | | Concentration of active | Kill rate in % |
|---|---|---|---|
| No. | Active compounds | compound in g/ha | after 7 d |
| 2 | 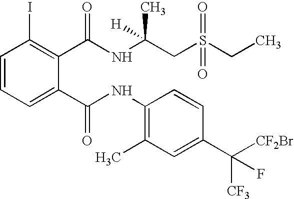 | 20 | 100 |
| 10 | 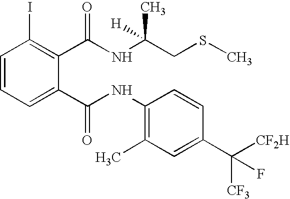 | 20 | 100 |
| 15 | 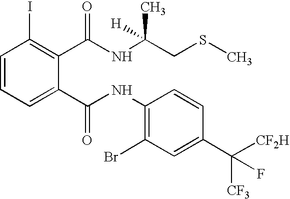 | 20 | 100 |
| 22 | 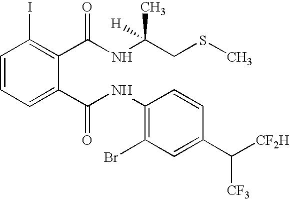 | 20 | 100 |
| 29 | 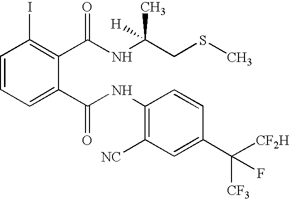 | 20 | 83 |
| 41 | 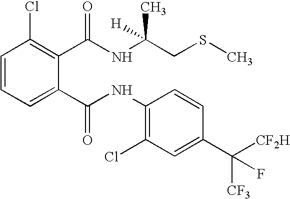 | 20 | 100 |
| 45 | 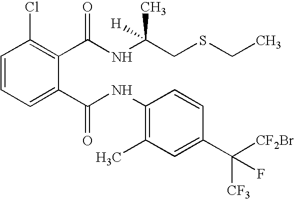 | 20 | 100 |
| 50 | 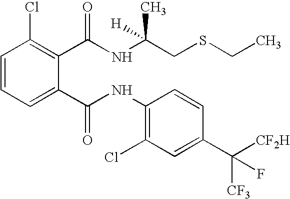 | 20 | 100 |
| 57 | 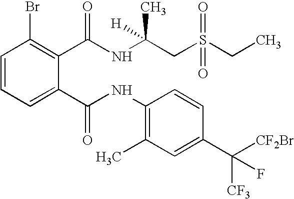 | 20 | 100 |
| 60 | 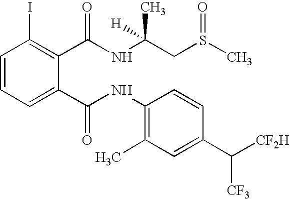 | 20 | 100 |
| 61 | 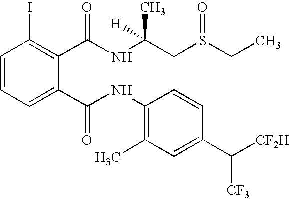 | 20 | 100 |

### Example G

### Plutella Test

| | |
|---|---|
| Solvent: | 7 parts by weight of dimethylformamide |
| Emulsifier: | 2 parts by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Cabbage leaves (Brassica oleracea) are treated by being dipped into the preparation of active compound of the desired concentration and are populated with caterpillars of the diamondback moth (Plutella xylostella) while the leaves are still moist.

After the desired period of time, the kill in % is determined. 100% means that all caterpillars have been killed; 0% means that none of the caterpillars have been killed.

In this test, for example, the following compounds of the preparation examples show good activity:

TABLE G
Plant-damaging insects
Plutella test

| | | Concentration of acitve | Kill rate in % |
|---|---|---|---|
| No. | Active compounds | compound in ppm | after 7 d |
| 11 | 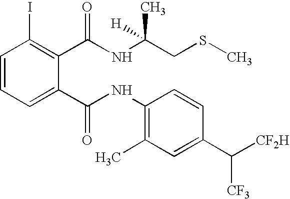 | 20 | 100 |
| 18 | 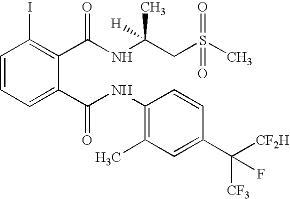 | 20 | 100 |
| 38 | 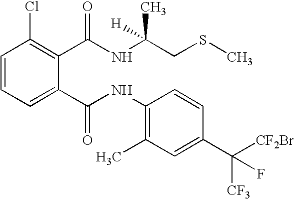 | 20 | 100 |
| 44 | 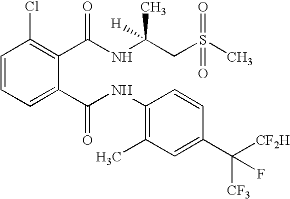 | 20 | 100 |
| 54 | 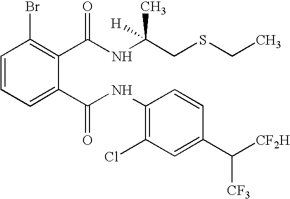 | 20 | 100 |
| 67 | 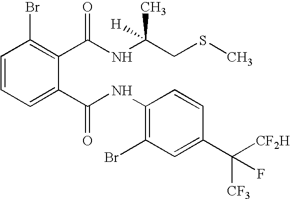 | 20 | 100 |

### Example H

### Spodoptera exigua Test

| | |
|---|---|
| Solvent: | 7 parts by weight of dimethylformamide |
| Emulsifier: | 2 parts by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Cabbage leaves (Brassica oleracea) are treated by being dipped into the preparation of active compound of the desired concentration and are populated with caterpillars of the army worm (Spodoptera exigua) while the leaves are still moist.

After the desired period of time, the kill in % is determined. 100% means that all caterpillars have been killed; 0% means that none of the caterpillars have been killed.

In this test, for example, the following compounds of the preparation examples show good activity:

TABLE H
Plant-damaging insects
Spodoptera exigua test

| | | Concentration of acitve | Kill rate in % |
|---|---|---|---|
| No. | Active compounds | compound in ppm | after 7 d |
| 8 | 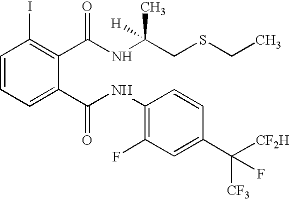 | 20 | 100 |
| 13 | 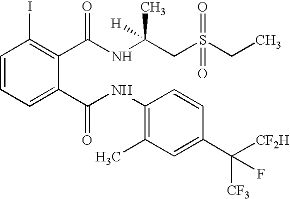 | 20 | 100 |
| 19 | 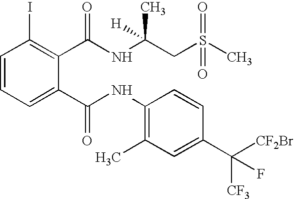 | 20 | 100 |
| 27 | 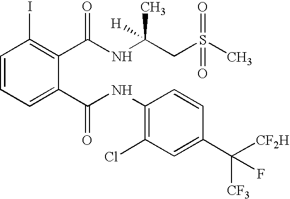 | 20 | 100 |
| 34 | 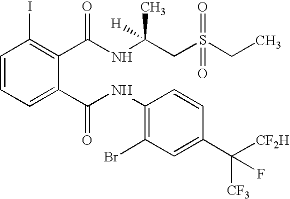 | 20 | 100 |
| 47 | 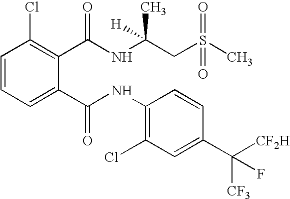 | 20 | 100 |
| 52 | 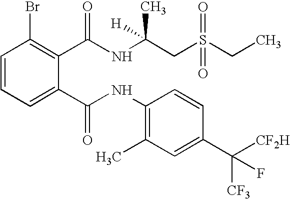 | 20 | 100 |
| 56 | 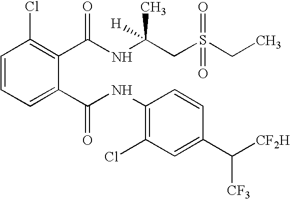 | 20 | 100 |
| 59 | 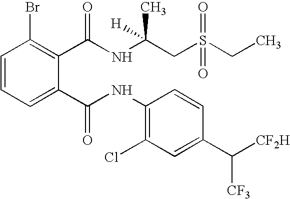 | 20 | 100 |

### Example I

### Spodoptera frugiperda Test (Spray Application)

| | |
|---|---|
| Solvent: | 78 parts by weight of acetone |
| | 1.5 parts by weight of dimethyl formamide |
| Emulsifier: | 0.5 parts by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Maize leave slices (Zea mays) are treated by being sprayed with the preparation of the active compound of the desired concentration and are infested with caterpillars of the fall army worm (Spodoptera frugiperda) after the leaves have dried.

After the specified period of time, the mortality in % is determined. 100% means that all the aphids have been killed; 0% means that none of the aphids have been killed.

In this test, for example, the following compounds of the preparation examples show good activity:

TABLE I
Plant-damaging insects
Spodoptera frugiperda test (Spray application)

| | | Concentration of active | Kill rate in % |
|---|---|---|---|
| No. | Active compounds | compound in g/ha | after 7 d |
| 6 | 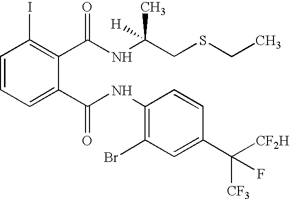 | 20 | 100 |
| 12 | 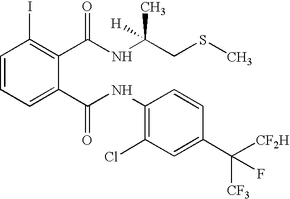 | 20 | 100 |
| 16 | 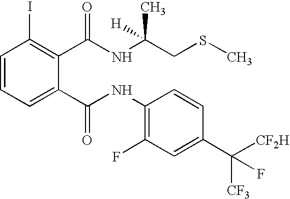 | 20 | 100 |
| 26 | 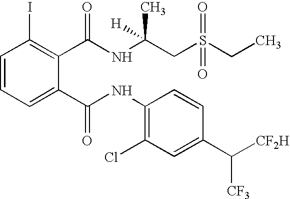 | 20 | 100 |
| 30 | 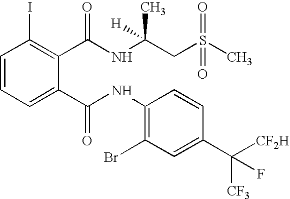 | 20 | 100 |
| 39 | 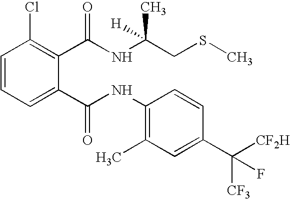 | 20 | 100 |
| 39 | 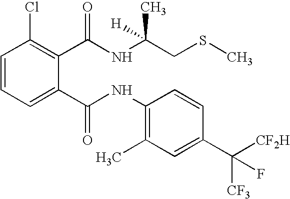 | 20 | 100 |
| 49 | 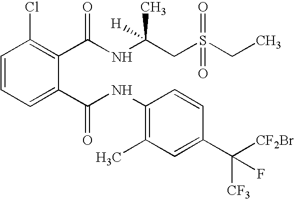 | 20 | 100 |
| 64 | 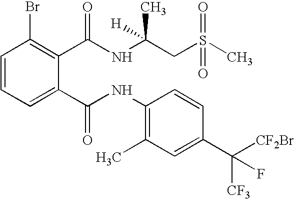 | 20 | 90 |
| 65 | 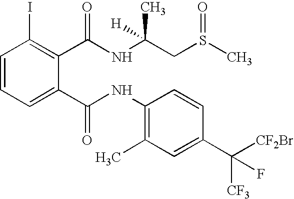 | 200 | 100 |
| 66 | 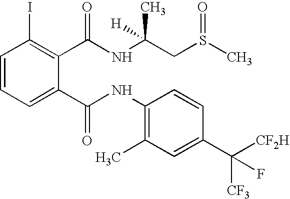 | 20 | 100 |
| 69 | 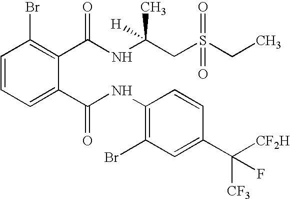 | 20 | 100 |

### Example J

### Tetranychus test (OP-Resistant/Spray Application)

| | |
|---|---|
| Solvent: | 78 parts by weight of acetone |
| | 1.5 parts by weight of dimethyl formamide |
| Emulsifier: | 0.5 parts by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration.

Bean leave slices (Phaseolus vulgaris), which are infested with all stages of the two-spotted spider mite (Tetranychus urticae), are treated by being sprayed with the preparation of the active compound of the desired concentration.

After the specified period of time, the mortality in % is determined. 100% means that all the aphids have been killed; 0% means that none of the aphids have been killed.

In this test, for example, the following compounds of the preparation examples show good activity:

TABLE J
Plant-damaging mites
Tetranychus test (OP-resistent/Spray application)

| | | Concentration of acitve | Kill rate in % |
|---|---|---|---|
| No. | Active compounds | compound in g/ha | after 5 d |
| 42 | 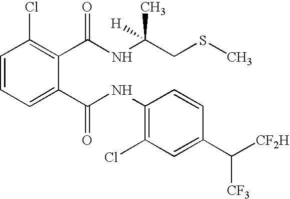 | 100 | 80 |
| 44 | 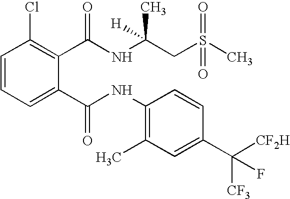 | 100 | 80 |
| 46 | 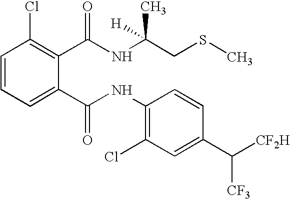 | 100 | 80 |

### Example K

### Diabrotica balteata Test (Larvae in Soil)

### Critical Concentration Test/Soil Insects—Treatment of Transgenic Plants

| | |
|---|---|
| Solvent: | 7 parts by weight of dimethylformamide |
| Emulsifier: | 1 part by weight of alkylaryl polyglycol ether |

To produce a suitable preparation of active compound, 1 part by weight of active compound is mixed with the stated amount of solvent, the stated amount of emulsifier is added and the concentrate is diluted with water to the desired concentration.

The preparation of active compound is poured onto the soil. Here, the concentration of active compound in the preparation is virtually irrelevant, only the amount by weight of active compound per volume unit of soil, which is stated in ppm (mg/l), matters. The soil is filled into 0.25 1 pots and these are allowed to stand at 20° C.

Immediately after preparation, 5 pre-germinated maize corns of the cultivar YIELD GUARD (trade mark of Monsanto Comp., USA) are placed into each pot. After 2 days, the test insects in question are placed into the treated soil. After a further 7 days, the efficacy of the active compound is determined by counting the maize plants that have emerged (1 plant=20% efficacy).

### Example L

### Heliothis virescens Test (Treatment of Transgenic Plants)

| | |
|---|---|
| Solvent: | 7 parts by weight of dimethylformamide |
| Emulsifier: | 1 part by weight of alkylaryl polyglycol ether |

To produce a suitable preparation of active compound, I part by weight of active compound is mixed with the stated amount of solvent and the stated amount of emulsifier, and the concentrate is diluted with water to the desired concentration.

Soybean shoots (Glycine max) of the cultivar Roundup Ready (trade mark of Monsanto Comp. USA) are treated by being dipped into the preparation of active compound of the desired concentration and are populated with the tobacco bollworm Heliothis virescens while the leaves are still moist.

After the desired period of time, the kill in % is determined. 100% means that all caterpillars have been killed; 0% means that none of the caterpillars have been killed.

1. An optically active compound of the formula (I) 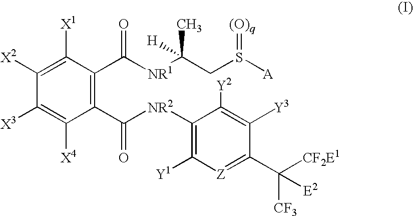
in which
A represents hydrogen, C₁-C₆-alkyl, C₃-C₆-alkenyl, C₃-C₆-alkynyl, C₁-C₆-alkoxy-C₁-C₄-alkyl, C₁-C₆-alkylthio-C₁-C₄-alkyl, C₁-C₆-alkylsulfinyl-C₁-C₄-alkyl, or (C₁-C₆-alkyl)-carbamoyl,
q represents 0, 1 or 2,
R¹ represents hydrogen or C₁-C₆-alkyl,
R² represents hydrogen or C₁-C₆-alkyl,
Z represents CY⁴ or N,
E¹ represents hydrogen or bromine,
E² represents hydrogen fluorine,
X¹, X², X³, X⁴ independently represent hydrogen, halogen, C₁-C₆-alkyl, C₁-C₆-haloalkyl, nitro, cyano, C₁-C₆-alkylsulfonyloxy, C₁-C₆ haloalkylsulfonyloxy, phenylsulfonyloxy, C₁-C₆-alkylthio-C₁-C₆-alkyl, C₁-C₆-alkylsulfinyl-C₁-C₆-alkyl, C₁-C₆-alkylsulfonyl-C₁-C₆-alkyl, C₁-C₆-alkylsulfonylamino, bis(C₁-C₆-alkylsulfonyl)amino or C₁-C₆-alkylcarbonyloxy,
Y¹, Y², Y³, Y⁴ independently represent hydrogen, halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio or cyano.

2. An optically active compound of the formula (I) according to claim 1, in which
A represents hydrogen, methyl, ethyl, n- or isopropyl, n-, iso-, sec- or tert-butyl, isomeric pentyl, isomeric hexyl, allyl, butenyl, pentenyl, hexenyl, propargyl, butynyl, pentynyl, hexynyl, C₁-C₄-alkoxy-C₁-C₂-alkyl, C₁-C₄-alkylthio-C₁-C₂-alkyl, C₁-C₄-alkylsulfinyl-C₁-C₂-alkyl, or (C₁-C₄-alkyl)carbamoyl,
q represents 0, 1 or 2,
R¹ represents hydrogen, methyl, ethyl, n- or isopropyl, n-, iso-, sec- or tert-butyl,
R² represents hydrogen, methyl, ethyl, n- or isopropyl, n-, iso-, sec- or tert-butyl,
Z represents CY⁴ or N,
E¹ represents hydrogen or bromine,
E² represents hydrogen or fluorine,
X¹, X², X³, X⁴ independently represent hydrogen, fluorine, chlorine, bromine, iodine, C₁-C₄-alkyl, C₁-C₄-haloalkyl, nitro, cyano, C₁-C₄-alkylsulfonyloxy, C₁-C₄-haloalkylsulfonyloxy, phenylsulfonyloxy, C₁-C₄-alkylthio-C₁-C₄-alkyl, C₁-C₄-alkylsulfinyl-C₁-C₄-alkyl, C₁-C₄-alkylsulfonyl-C₁-C₄-alkyl, C₁-C₄-alkylsulfonylamino, bis(C₁-C₄-alkylsulfonyl)amino or C₁-C₄-alkylcarbonyloxy,
Y¹, Y², Y³, Y⁴ independently represent hydrogen, fluorine, chlorine, bromine, iodine, C₁-C₄-alkyl, C₁-C₄-haloalkyl, C₁-C₄-alkoxy, C₁-C₄-haloalkoxy, C₁-C₄-alkylthio, C₁-C₄-haloalkylthio or cyano.

3. A process for preparing a compound of the formula (I) according to claim 1 comprising:
1) a) reacting a compound of formula (IV) 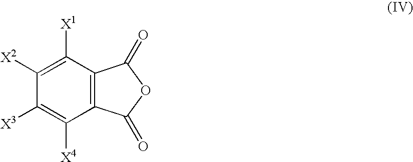
with a compound of formula (V) 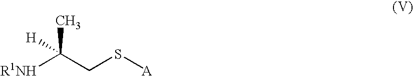
to obtain a product of formula: 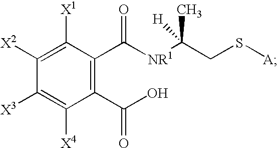
b) reacting the product of step (a) with a condensing agent to obtain a product of formula: 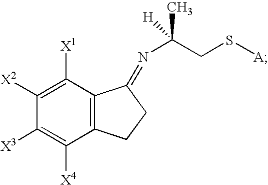
c) reacting the product of step (b) with a compound of formula (II): 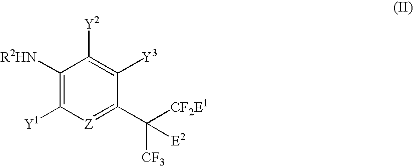
to obtain a product of formula: 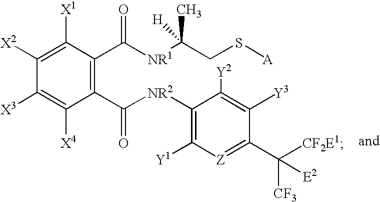
d) reacting said product of step (c) with an oxidizing agent to obtain a compound of formula (I);
or
2) a)′ reacting a compound of formula (IV) 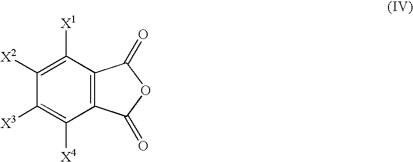
with a compound of formula (II) 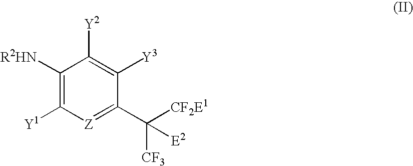
to obtain a product of formula: 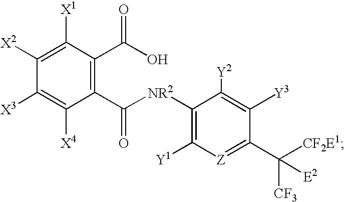
b)′ reacting the product of step (a)′ with a condensing agent to obtain a product of formula: 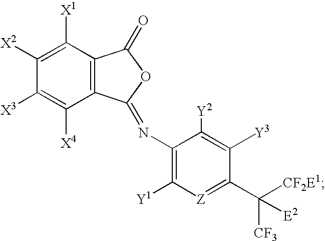
c)′ reacting the product of step (b)′ with a compound of formula (V): 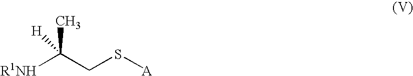
to obtain a product of formula: 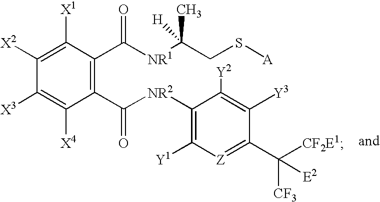
d)′ reacting said product of step (c)′ with an oxidizing agent to obtain a compound of formula (I);
in which
A, q, R¹, R², Z, E¹, E², X¹, X², X³, X⁴, Y¹, Y², Y³, and Y⁴ are as defined in claim 1.

4. A pesticide comprising at least one compound of the formula (I) according to claim 1, and one or more extenders or surfactants, or combinations thereof.

5. (canceled)

6. A method for controlling pests, comprising contacting said pests or their habitat with a compound of the formula (I) according to claim 1.

7. A process for preparing a pesticide, comprising mixing a compound of formula (I) according to claim 1 with one or more extenders or surfactants, or combinations thereof.

8. A compound of formula (II) 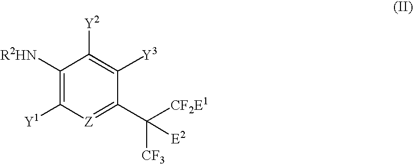
in which
R² represents hydrogen or C₁-C₆-alkyl,
Z represents CY⁴ or N,
E¹ represents hydrogen or bromine,
E² represents hydrogen or fluorine,
Y¹, Y², Y³, Y⁴ independently represent hydrogen, halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio or cyano.

9. A process for preparing a compound of formula (II) according to claim 8 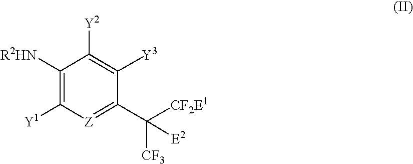
wherein E¹ and E² are hydrogen,
comprising:
(a) reacting a compound of formula (III) 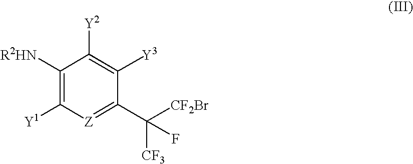
with tributyltin hydride to obtain a product of formula: 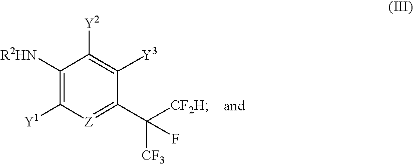
(b) reacting the product of step (a) with sodium borohydride to obtain a product of formula: 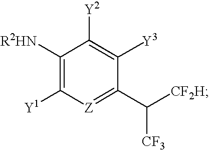
in which
R², Z, Y¹, Y² and Y³ are as defined in claim 8.
